(19) United States
(12) Patent Application Publication
(10) Pub. No.: US 2011/0275699 A1
(43) Pub. Date: Nov. 10, 2011

(54) Treatment For Obesity And Diabetes

(75) Inventors: Hei Sook Sul, Berkeley, CA (US); Roger Hoi Fung Wong, Berkeley, CA (US)

(73) Assignee: University of California

(21) Appl. No.: 13/048,637

(22) Filed: Mar. 15, 2011

Related U.S. Application Data
(60) Provisional application No. 61/314,435, filed on Mar. 16, 2010.

(51) Int. Cl. A61K 31/7105; A61P 3/04; A61P 3/00; A61P 13/12; A61P 3/10

(52) U.S. Cl. 514/44 A

(57) ABSTRACT

The present disclosure relates to strategies aimed at treating/preventing obesity and diabetes. In particular, obesity is a major public health problem, associated with detrimental metabolic consequences such as diabetes, cardiovascular disease, stroke, osteoarthritis and even some types of cancer. Thus, application of DNA-PK inhibitors, that has been connected to the signaling pathway involved in the formation of fat from carbohydrate in the liver, could potentially be a pharmacological target for regulation of obesity and diabetes due to a diet high in carbohydrates. Therefore, the invention finds application in the fields of obesity, diabetes, and lipogenesis research and therapy.

### CROSS-REFERENCE TO RELATED APPLICATIONS

The present application claims the benefit of U.S. Provisional Patent Application No. 61/314,435, filed on. Mar. 16, 2010, which is incorporated herein by reference.

### STATEMENT REGARDING FEDERALLY SPONSORED RESEARCH OR DEVELOPMENT

The research described in this application was supported by NIH funding, grant numbers DK9198 (LR01 DK081098) and DK75682 (R01 DK075682). The United States government might have certain rights in the invention.

### FIELD OF THE INVENTION

The invention finds application in the fields of obesity, diabetes, and lipogenesis research and therapy. More specifically, the invention provides targets and inhibitors for treating and/or preventing obesity, kidney disease, and metabolic diseases such as diabetes.

### BACKGROUND OF THE INVENTION

To meet the constant energy requirement in the face of highly variable food supply, mammals employ intricate and precise mechanisms for energy storage. When total energy intake is in excess of energy expenditure such as after a meal, excess carbohydrates are converted to fatty acids (de novo lipogenesis). Excess fatty acids are then converted to triacylglycerol to be stored in adipose tissue and released as oxidative fuels for other tissues during times of energy need such as fasting and exercise. In sustaining the balance between energy excess and energy deficiency, the process of lipogenesis is tightly controlled by nutritional and hormonal conditions. (Sul et al. 2008). Thus, enzymes involved in fatty acid and fat synthesis are tightly and coordinately regulated during fasting/feeding: Activities of these enzymes are very low in fasting due to the increase in glucagon/cAMP levels. Conversely, in the fed condition, especially after a high carbohydrate meal, activities of these enzymes drastically increase as blood glucose and insulin levels rise. (Sul et al. 2008). Fatty acid synthase (FAS), a central lipogenic enzyme, plays a crucial role in de novo lipogenesis by catalyzing all of the seven reactions involved in fatty acid synthesis. While FAS is not known to be regulated by allosteric effectors or by covalent modification, its transcription is exquisitely regulated by fasting/feeding and by insulin. (Sul et al. 2008). The FAS promoter thus provides an excellent model system to dissect the transcriptional activation of lipogenesis by feeding/insulin.

In early studies of insulin regulation of the FAS promoter, it was found that Upstream Stimulatory Factor (USF) binding to the −65 E-box is required for transcriptional activation by insulin. (Wang et al. 1995; Wang et al. 1997; and Griffin et al. 2007). The critical role of USF in the activation of the FAS promoter by insulin was further verified by overexpressing dominant negative or wild type forms of USF. (Wang et al. 1995; and Wang et al. 1997). The induction of FAS by fasting/feeding was significantly impaired in USF knockout mice. (Casado et al. 1999). Functional analysis and chromatin immunoprecipitation (ChIP) in mice transgenic for various 5′ deletions and mutations of the FAS promoter-CAT reporter gene showed that USF binding to the −65 E-box is required for feeding/insulin-mediated FAS promoter activation in vivo. (Latasa et al. 2003). Notably, USF binding was detected in both fasted and fed states. On the FAS promoter, USF recruits another transcription factor SREBP-1, whose level increases upon insulin treatment via the PI3K pathway, (Wang et al. 1998) to bind the −150 SRE and mediate insulin/feeding responsiveness. (Latasa et al. 2000). Although early studies of ectopically expressed SREBP-1 in cultured cells has been shown to bind the −65 E-box, (Kim et al. 1998) the functional analysis and chromatin immunoprecipitation (ChIP) in mice transgenic for various 5′ deletions and mutations of the FAS promoter-CAT reporter gene clearly showed SREBP-1 binds the −150 SRE, but not −65 E-box to activate the FAS promoter during feeding/insulin treatment in vivo. (Latasa et al. 2003). Although SRBEP-1c binding to the −150 SRE is critical for the feeding/insulin response, SREBP-1c itself cannot bind the SRE without being recruited by USF, which is constitutively bound to the −65 E-box. (Griffin et al. 2007; Latasa et al. 2003; and Wong et al. 2009). Many lipogenic promoters contain a closely spaced E-box and SRE in the proximal promoter region, and a similar mechanism for activation of several lipogenic genes has been documented previously. (Griffin et al. 2007). Possibly, the SREBP-1c promoter is also regulated by USF and SREBP-1c in response to feeding/insulin. Thus, USF, along with SREBP-1c, plays a critical role in mediating the transcriptional activation of lipogenesis in response to feeding/insulin.

Studies have shown that LXR may play a role in the transcriptional regulation of lipogenesis by activating SREBP-1c transcription. (Repa et al. 2000). LXR has also been reported to directly regulate the FAS promoter in cultured cells. (Joseph et al. 2002). A carbohydrate response element (ChoRE) where ChREBP can bind has also been reported to be present far upstream of the FAS promoter region. (Ishii et al. 2004). Nevertheless, FAS promoter-reporter transgenic mice studies showed that the FAS promoter that contains both an E-box and SRE, but lacks a LXRE or ChoRE, is sufficient for high-level activation of the FAS promoter during fasting/feeding suggesting that binding of LXR or ChREBP may not be critical in vivo. (Latasa et al. 2003). Regardless, questions remain in understanding the FAS promoter activation involving USF and SREBP. Some of the other issues, apart from HAT/HDAC, include are other coactivators required for activation; what chromatin remodeling machinery and mediators are recruited to the FAS promoter; are there common mechanisms to explain the transcriptional regulation of other coordinately regulated lipogenic genes; and is chromatin folding involved in sharing transcription machineries among lipogenic gene promoters. Thus, further studies are necessary to understand the details of the transcriptional activation of lipogenic genes. While many metabolic effects of insulin are mediated through protein phosphorylation via the well characterized PI3K cascade, which activates PKB/Akt, insulin can also exert metabolic effects through dephosphorylation catalyzed mainly by PP1. (Brady et al. 2001). Regardless, USF is bound to the E-box on the FAS promoter in both fasted and fed states and neither USF expression nor post-translational modification have been shown to be altered by insulin. Although it is suggested that USF mediates the insulin response of lipogenic gene promoters, the precise mechanism of how USF responds to insulin is not fully understood.

### SUMMARY OF THE INVENTION

Obesity is a major public health problem, associated with detrimental metabolic consequences such as diabetes, cardiovascular disease, stroke, osteoarthritis and even some types of cancer. Over thirty percent of Americans are obese and over sixty percent are overweight. Similarly, the American population with insulin-resistant or Type 2 diabetes is growing rapidly. Therefore, strategies aimed at treating/preventing obesity and diabetes are crucial for prevention of these diseases. However, recommendations to eat less and exercise more have proven to be ineffective and current therapeutics have been unsuccessful, often because of undesirable side effects. The present invention describes for the first time that DNA-PK is connected to the signaling pathway involved in the formation of fat from carbohydrate in the liver. Therefore, DNA-PK is a pharmacological target for regulation of obesity and diabetes due to a diet high in carbohydrates. The invention finds application in the fields of obesity, diabetes, and lipogenesis research and therapy. More specifically, the invention provides targets and inhibitors for treating and/or preventing (or simply slowing the progression of) obesity, kidney disease and metabolic diseases such as diabetes.

In some embodiments the present invention provides a method for treating a subject for kidney disease, comprising providing i) a mammalian subject in need of treatment for a kidney disease; ii) a DNA-PK inhibitor; and b) administering to said subject said inhibitor under conditions to treat said disease. In another embodiment, the present invention provides a method for treating symptoms of diabetes comprising, providing i) a mammalian subject exhibiting symptoms of diabetes; ii) a DNA-PK inhibitor; and b) administering to said subject said inhibitor in an amount where at least one symptom is reduced.

In further embodiments, the present invention provides a composition, comprising a DNA-PK inhibitor. In other embodiments, the invention provides a method for treating a subject for metabolic disease, comprising providing i) a mammalian subject in need of treatment for a metabolic disease; ii) a DNA-PK inhibitor; and b) administering to said subject said inhibitor under conditions to treat said disease.

In other embodiments the present invention provides a method for treating symptoms of obesity comprising, providing i) a mammalian subject exhibiting symptoms of obesity; ii) a DNA-PK inhibitor; and b) administering to said subject said inhibitor in an amount where at least one symptom is reduced. In another embodiment the present invention provides a method for treating kidney disease, comprising providing i) a mammalian subject in need of treatment for a kidney disease; ii) a siRNA construct targeted to DNA-PK; and b) administering said construct to said subject in an amount to treat said disease.

While specific embodiments are given it is optionally desirable for other things to be used that are known in the state of the art. Further, compositions given might optionally include a pharmaceutically acceptable carrier and the embodiments can be used alone or in other formulations as known in the art of drug delivery. In further embodiments the present invention contemplates use of the inhibition of DNA-PK as a method for screening and identifying small molecules of use for treating obesity, diabetes, metabolic disease, kidney disease, and other related diseases. Further, other embodiments might contemplate methods and/or compositions for treating cancer, heart disease, and other related conditions to obesity, diabetes, kidney disease, and/or metabolic disease. Moreover, while siRNA is specifically mentioned it is also contemplated that other techniques are encompassed such as use of miRNA, hairpin siRNA, ds siRNA, and equivalents known in the art including use of antibodies and equivalents. In addition, other DNA-PK inhibitors are known in the art such as those found in the following publications, which are herein incorporated by reference in their entirety Kashishian et al. Mol. Cancer Therapeutics, Dec. (2)12:1257-1264 (2003); Durant et al. Nucleic Acids Research, 31 (No. 19):5501-5512 (2003); U.S. Pat. Nos. 7,226,918; 7,402,607; and 7,674,823; and U.S. Patent Application Publication's 2007/0238729; 2004/0192687; 2006/0106025; 2006/0264427; 2006/0264623; 2007/0238731; 2008/0038277 and 2009/0042865 which are herein incorporated by reference in their entirety. While specific cells, examples, reagents, methods, and sequences are given they are not meant to be limiting and include other known comparable techniques in the state of the art.

### BRIEF DESCRIPTION OF THE DRAWINGS

FIG. 1A-H demonstrates purification of USF-1-Interacting proteins where (A) The identities (far left) of USF-1-associated polypeptides. Purified USF-1 eluates on SDS-PAGE by silver staining (second from left). Immunoblotting of TAP eluates (middle). IP of USF-1 (second from right) from 293F cells with monoclonal anti-USF-1 antibodies. TAP eluates from 293F cells it was re immunoblotted (far right). (B) RNA from tissues was used for RT-PCR. (C) ChIP for association of USF-1-interacting proteins to the −444 FAS-CAT promoter (left) in FAS-CAT transgenic mice or the mGPAT promoter (right) in WT mice. (D) Expression in liver determined by RT-qPCR. (E) IP of FLAG-tagged USF-1 from HepG2 cells. (F) ChIP for association of USF-1-interacting proteins to the −444 (−65 m) FAS-CAT (left) promoter or the FAS promoter in HepG2 cells (right). USF-1 protein levels by immunoblotting (bottom right). (G) ChIP for binding of USF-1-interacting proteins to the −444 FAS-CAT (left) and −444 (−150 m) FAS-CAT (right) promoter. (H) ChIP analysis for DNA breaks and DNA-PK and TopoIIβ binding to the FAS-CAT (left) or the endogenous FAS promoter (right) in FAS-CAT transgenic or wildtype mice. Error bars represent ±SEM.

FIG. 2A-F demonstrates feeding-induced S262 phosphorylation and K237 acetylation of USF-1 where (A) USF-1 immunoprecipitates using monoclonal anti-USF-1 was western blotted with polyclonal anti-USF-1 or anti-P-USF-1. Immunoblotting with anti-P-USF-1 in the presence of peptide or with preimmune serum are shown as controls. (B) ChIP for indicated proteins binding to the −444 FAS-CAT promoter. (C) ChIP (top) for WT USF-1 and S262 USF-1 mutant association to the FAS promoter in 293FT cells. The FAS promoter activity (bottom) was monitored. Immunoblotting for protein levels of WT, S262 USF-1 mutants (insert), and FAS are shown. (D) IP of USF-1. (E) ChIP for binding of indicated proteins to the −444 FAS-CAT promoter. (F) ChIP (top) for association of WT USF-1 and K237 USF-1 mutant to the FAS promoter. The promoter activity was measured. Error bars represent ±SEM.

FIG. 3A-H demonstrates feeding-dependent S262 phosphorylation of USF-1 is mediated by DNA-PK that is dephosphorylated/activated in feeding where (A) USF-1 was incubated with DNA-PK. (B) IP of USF-1. Immunoblotting for DNA-PK. (C) EP (left) of USF-1. The FAS promoter activity was measured (right). (D) DNA-PK activity was assayed. (E) IP of DNA-PK. (F) IP of USF-1-FLAG. Total and phosphorylated DNA-PK by it was stern blotting. (G) IP of USF-1. PP1 protein levels by western blotting. (H) IP of PP1 from nuclear extracts or total lysates. USF-1 and beta-actin protein levels by western blotting. Error bars represent ±SEM.

FIG. 4A-F demonstrates acetylation of K237 of USF-1 by P/CAF and deacetylation by HDAC9 where (A) IP of USF-1 (top). USF-1 was in vitro acetylated with P/CAF (bottom). (B) IP of USF-1. P/CAF protein levels by immunoblotting. (C) IP of USF-1. (D) USF-1 was incubated with in vitro translated ³⁵S-labeled proteins before subjecting to GST pull-down. GST was used as a control. (E) The −444 FAS-Luc promoter activity was measured. (F) The −444 FAS-Luc promoter activity was measured. Total cell lysates were immunoblotted. Error bars represent ±SEM.

FIG. 5A-K demonstrates feeding/insulin-induced phosphorylation and acetylation of USF-1 are greatly reduced in DNA-PK deficiency where (A-C) IP of FLAG-tagged USF-1. Nuclear extracts from nontransfected cells were used as a control. (D) IP of USF-1-FLAG from HepG2 cells (top). Total protein levels by immunoblotting (bottom). (E and F) IP of USF-1 from HepG2 cells (E) or from M059J or M059K cells (F). (G) ChIP for binding of indicated proteins to the FAS promoter. (H) IP of USF-1. (I) ChIP for indicated protein association to the FAS promoter. ChIP samples were analyzed by semiquantitative PCR (top) or qPCR (bottom). (J) IP of USF-1. (K) ChIP for indicated protein association to the FAS promoter. Error bars represent ±SEM.

FIG. 6A-H demonstrates diminished FAS induction leading to blunted de novo lipogenesis and decreased triglyceride levels in liver and serum where (A and B) Nascent RNA were used for (A) RT-PCR or (B) RT-qPCR. Fold induction normalized by β-actin. (C) Run-ons of labeled nascent transcripts were analyzed by RT-qPCR. (D) ChIP for DNA breaks and indicated protein binding to the FAS promoter. (E) Newly synthesized labeled fatty acids in livers from 9-week-old mice were measured. Values are means±SEM. n=12. (F) Immunoblotting of equal amounts of liver extracts from 9-week-old mice after 24 hr of feeding. (G) Hepatic and serum triglyceride levels were measured in 9-week-old fed mice. (H) Schematic representation of USF-1 and its interacting partners and their effects on lipogenic gene transcription in fasting/feeding. Error bars represent ±SEM.

FIG. 7A-B shows WT compared to SCID mice for body weight.

FIG. 8A-E shows (A) Bacterially expressed USF-1-GST fusion protein was incubated with various in vitro translated ³⁵S-labeled interacting proteins individually before subjection to GST pulldown and autoradiography. Purified USF-1 complex was subjected to autoradiography (left) for labeled proteins. GST protein alone was used as a control. USF-1-GST fusion protein was incubated with purified recombinant PARP-1 or PP1 before subjection to GST pull-down and immunoblotting (top right) for indicated proteins. GST and GSTUSF proteins after SDS-PAGE were stained with Coomassie blue (bottom right). (B) Detection of USF-1 complex: TAP eluates were separated by BN-PAGE and then subjected to Western blotting with anti-FLAG for USF-1. In lanes 2 and 3, supershift assays were carried out with 2 μg polyclonal anti-USF-1 (lanes 2) or 2 μg control polyclonal anti-GAPDH (lane 3). A minor faster migrating complex observed was probably due to partial dissociation of USF-1 interacting proteins from the complex during the sample preparation. (C) A representative ChIP for USF-1 interacting protein association to the p53 promoter in liver (left). p53 gene expression in livers from fasted or fed mice determined by RTqPCR (right). (D) IP of USF-1 interacting proteins from HepG2 cells treated with or without insulin at 100 nM for 30 min. Immunoprecipitates were Western blotted for each of the USF-1 interacting proteins. (E) ChIP analysis for biotin incorporation into 3′-ends of DNA breaks and DNA-PK and TopoIIβ binding to the control FAS coding region in livers from fasted or fed FAS-CAT transgenic or wild type mice.

FIG. 9A-E shows (A) ChIP for binding of various USF-1 interacting proteins to the −444 FAS-CAT (left) and −444(−150 m) FAS-CAT (right) promoter regions in livers from fasted or fed FASCAT transgenic mice. (B) ChIP for binding of USF-1 interacting proteins to the −444 FAS-Luc in HepG2 cells transfected with control or SREBP-1 siRNA. Cells were treated with or without 100 nM insulin for 30 min. SREBP-1 protein levels analyzed by Western blotting are shown (bottom). (C) Bacterially expressed SREBP-1-GST fusion protein was incubated with various in vitro translated ³⁵S-labeled interacting proteins individually before subjection to GST pull-down and autoradiography. Purified SREBP-1 complex was subjected to autoradiography for labeled proteins. GST protein alone was used as a control.

(D) ChIP for binding of various USF-1 interacting proteins to the −700 p53-Luc and −700 (SRE) p53-Luc promoter regions (SRE was inserted 89 bases upstream of the proximal E-box of the p53 promoter, by substitution of CCTCAACCCAC [SEQ ID. NO. 41] to CATCACCCCAC [SEQ ID. NO. 42]) in
HepG2 cells treated with or without 100 nM insulin for 30 min (left). ChIP (right) for binding of USF-1 interacting proteins to the −700 (SRE) p53-Luc in HepG2 cells transfected with control or SREBP-1 siRNA. (E) The FAS promoter activity in cells transfected with the −444 FAS-Luc or −444 (−150 m) FAS-Luc along with SREBP-1 siRNA was measured by luciferase reporter assay (left). The p53 promoter activity in cells transfected with the −700 p53-Luc or −700 (SRE) p53-Luc along with SREBP-1 siRNA was measured by luciferase reporter assay (right).

FIG. 10A-E shows (A) Bacterially expressed USF-1incubated with varying concentrations of DNA-PK was immunoblotted for total USF-1 and P262S USF-1. (B) Bacterially expressed USF-1 was incubated with DNA-PK, PKA, or PKC. Reaction mixtures were subjected to Western blotting for total USF-1 and P262S USF-1 (top). IP of 293F cells overexpressing USF-1 and PKB-HA with anti-FLAG antibodies. USF-1 immunoprecipitated with FLAG antibodies was Western blotted for total USF-1 and P262S USF-1 (bottom). PKB levels were analyzed by western blotting with anti-HA antibodies as shown. (C) Levels of total DNA-PK and phosphorylated DNA-PK in cells overexpressing DNAPK with empty vector or PP1 expression vector were analyzed by immunoblotting. (D) IP of cells cotransfected with USF-1, control or PP1 siRNA, and DNA-PK mutants. Immunoprecipitated USF-1 was Western blotted for total USF-1 and P262S USF-1. (E) PP1 gene expression in livers from fasted or fed mice was determined by RT-qPCR.

FIG. 11A-E shows (A) IP of liver nuclear extracts with anti-HDAC9 antibodies. Immunoprecipitates were Western blotted for HDAC9. Immunoprecipitated HDAC9 from nuclear extracts of HepG2 cells treated with or without insulin at 100 nM for 30 min was Western blotted for HDAC9. USF-1 and β-actin protein levels were analyzed by Western blotting. (B) HDAC9 gene expression in livers from fasted or fed mice was determined by RTqPCR. (C) IP of cells transfected with USF-1 using polyclonal anti-USF-1 antibodies. Immunoprecipitated USF-1 was Western blotted (left) for USF-1 and p300. Normal IgG was used as a control. Bacterially expressed USF-1 GST fusion protein was incubated with in vitro translated ³⁵S-labeled P/CAF or HDAC9 before subjecting to GST-pull down. Purified USF-1 was subjected to autoradiography (right) for labeled proteins. GST alone was used as a control. (D) Total cell lysates in cells transfected with the −444 FAS-Luc, USF-1, and increasing amount of P/CAF or HDAC9 were Western Blotted for p53 protein levels (E) FAS promoter activity in cells transfected with the −444 FAS-Luc and WT, K237A or K237R USF-1, along with P/CAF or HDAC9 was measured (top). Western blotting of total cell lysates for FAS protein levels (bottom).

FIG. 12A-E shows (A) IP of nuclear extracts from M059J or M059K cells with anti-USF-1 antibodies and subsequent Western blotting for total USF-1, and various USF-1 interacting proteins. Cells were treated with, or without, 100 nM insulin for 30 min. (B) IP of USF-1-FLAG from 293 cells overexpressing USF-1 treated with either control DMSO and Taut at indicated concentrations for 2 hrs. USF-1 immunoprecipitated with FLAG antibodies was Western blotted for total USF-1 and various USF-1 interacting proteins. (C) ChIP for binding of USF-1 and its interacting proteins to the FAS promoter in 293 cells that were treated with either control DMSO and Taut at indicated concentrations for 2 hrs. (D) ChIP for P262S USF-1 and total USF-1 association to the mGPAT promoter in livers from WT and SCID mice. (E) TAP-eluates from cells overexpressing USF-1-TAP was subjected to Western blotting using anti-poly(ADP-ribosyl)ated antibodies. Empty TAP vector was used as control.

FIG. 13 shows WT (wild-type) compared to SCID mice for total oxygen consumption.

### DEFINITIONS

To facilitate the understanding of this invention a number of terms (set off in quotation marks in this Definitions section) are defined below. Terms defined herein (unless otherwise specified) have meanings as commonly understood by a person of ordinary skill in the areas relevant to the present invention.

As used herein, the term “polymerase chain reaction” (“PCR”) refers to the method of K. B. Mullis U.S. Pat. Nos. 4,683,195, 4,683,202, and 4,965,188, hereby incorporated by reference, that describe a method for increasing the concentration of a segment of a target sequence in a DNA mixture without cloning or purification. Because the desired amplified segments of the target sequence become the predominant sequences (in terms of concentration) in the mixture, they are said to be “PCR amplified.” Similarly, the term “modified PCR” as used herein refers to amplification methods in which a RNA sequence is amplified from a DNA template in the presence of RNA polymerase or in which a DNA sequence is amplified from an RNA template the presence of reverse transcriptase.

The term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, Winter and Milstein, Nature, 349, 293-299, 1991). As used herein, the term “antibody” encompasses recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab).sub.2 fragments. The term “reactive” when used in reference to an antibody indicates that the antibody is capable of binding an antigen of interest. In one embodiment a DNA-PK reactive antibody is contemplated.

The term “overexpression” or “overexpressed” refers to the production of a gene product in transgenic organisms that exceeds levels of production in normal or non-transformed organisms.

As used herein the term “nucleic acid sequence” refers to an oligonucleotide, a nucleotide or a polynucleotide, and fragments or portions thereof, and vice versus, and to DNA or RNA of genomic or synthetic origin, which may be single or double-stranded, and represent the sense or antisense strand. Similarly, “amino acid sequence” as used herein refers to peptide or protein sequence.

The term “antisense” when used in reference to DNA refers to a sequence that is complementary to a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex.

The term “RNA interference” or “RNAi” refers to the silencing of a gene wherein the translation of a gene is down regulating or decreasing of gene expression by RNAi molecules (e.g., siRNAs, miRNAs). It is the process of sequence-specific, post-transcriptional gene silencing in animals and plants, initiated by RNAi molecules that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial. In one embodiment, an siRNA construct is contemplated that either completely or partially inhibits the function of the DNA-PK gene.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, bacterial infection, viral infection, biolistics (i.e., particle bombardment) and the like.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is that which is most frequently observed in a population and is thus arbitrarily designated the “normal” or “wild-type” form of the gene.

In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The terms “mammals” and “mammalian” refer animals of the class mammalia, which nourish their young by fluid secreted from mammary glands of the mother, including human beings. The class “mammalian” includes placental animals, marsupial animals, and monotrematal animals. An exemplary “mammal” may be a rodent, primate (including simian and human) ovine, bovine, ruminant, lagomorph, porcine, caprine, equine, canine, feline, ave, etc. Preferred non-human animals are selected from the order Rodentia.

The terms “Western blot analysis” and “Western blot” and “Western” refers to the analysis of protein(s) (or polypeptides) immobilized onto a support such as nitrocellulose or a membrane. A mixture comprising at least one protein is first separated on an acrylamide gel, and the separated proteins are then transferred from the gel to a solid support, such as nitrocellulose or a nylon membrane. The immobilized proteins are exposed to at least one antibody with reactivity against at least one antigen of interest. The bound antibodies may be detected by various methods, including the use of radiolabeled antibodies.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein the term “treatment”, refers to any and all uses which remedy a disease state or symptoms, prevent the establishment of disease, or otherwise prevent, hinder, retard, or reverse the progression of disease or other undesirable symptoms in any way whatsoever. It is not limited to the case where the disease is cured. In some embodiments, treatment simply reduces one or more symptoms or simply slows the progression of disease.

In one embodiment, the present invention contemplates reducing one or more symptom of obesity. Such symptoms include extra fat around the waist, a higher than normal body mass index and waist circumference, weight gain, and clothes fitting tighter and needing a larger size among others. Furthermore, being obese increases the risk of diabetes, heart disease, stroke, arthritis, high blood pressure, asthma, gallstones, cholesterol and triglyceride problems, liver problems, digestive disorders, and some cancers.

In one embodiment, the present invention contemplates reducing one or more symptom of metabolic disease. Metabolic diseases include diabetes, amyloidosis, acid lipase disease, lipid storage disease, mitochondrial myopathies, Type I glycogen storage disease, and Farbers disease among others. Symptoms of metabolic disease include buildup of toxic fats due to lack of or missing enzyme for breakdown of fats for lipase disease; lack of growth, enlarged liver, chronic hunger, and fatigue among others for Type I glycogen storage disease; enlarged tongue, severe fatigue, shortness of breath, irregular heartbeat, protein in the urine, and tingling in hands and feet among others for amyloidosis; frequent urination, extreme hunger, extreme fatigue and irritability, blurred vision, tingling/numbness in hands/feet, recurring skin, gum, or bladder infections, cuts/bruises that are slow to heal, frequent infections, unusual thirst, and unusual weight loss among others for Type 1 and/or Type 2 diabetes; arthritis, swollen lymph nodes and joints, hoarseness, nodules under the skin (and sometimes in the lungs and other parts of the body), chronic shortening of muscles or tendons around joints, and vomiting among others for Farbers disease; muscle weakness or exercise intolerance, heart failure or rhythm disturbances, dementia, movement disorders, stroke-like episodes, deafness, blindness, droopy eyelids, limited mobility of the eyes, vomiting, and seizures among others for mitochondrial myopathies.

In one embodiment, the present invention contemplates reducing one or more symptom of kidney disease. Kidney disease is characterized by damage to the nephrons, which may leave kidneys unable to remove wastes. The damage usually occurs slowly over several years and there are no obvious symptoms, so it is hard to know that it is happening. Many things can cause kidney disease, and one is at risk if you have diabetes, high blood pressure, and/or a family member with kidney disease. Symptoms of kidney disease include changes in urination such as more frequent urination and/or pale urine, less frequent urination or in smaller amounts with dark colored urine, the urine may contain blood, the urine may be foamy or bubbly; swelling since failing kidneys don't remove extra fluid, which might build up in the body such as feet, legs, ankles, face, and/or hands; fatigue; skin rash/itching since removal of waste is compromised; metallic taste in mouth/ammonia breath because of buildup of wastes in the blood, which can make food taste different and cause bad breath; nausea and vomiting; shortness of breath due to fluid buildup and anemia because of a reduction in red blood cells that carry oxygen; feeling cold; dizziness and trouble concentrating; and leg/flank pain.

In one embodiment, the present invention contemplates reducing one or more symptom of diabetes. Such symptoms include frequent urination, unusual thirst, extreme hunger, unusual weight loss, and extreme fatigue and irritability for Type 1. Type 2 might have no symptoms and/or any of the Type 1 symptoms, frequent infections, blurred vision, cuts/bruises that are slow to heal, tingling/numbness in the hands/feet, recurring skin, gum, or bladder infections.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “stable isotope” refers to a chemical isotope that is not radioactive i.e. has not been observed to decay although some might still decay but have not been detected due to an extremely long half-life.

As used herein, the term “de novo lipogenesis” refers to the production and accumulation of fat. Fat production also includes either fatty degeneration or fatty infiltration and can be applied to the normal deposition of fat or to the conversion of carbohydrate or protein to fat (Stedman's Medical Dictionary, 26^{th} Ed., 1995). More particularly, the process of generating fatty acids from excess carbohydrates for synthesis/storage of triacylglycerol that can be utilized during an energy shortage such as fasting.

As used herein, the term “chromatin immunoprecipitation” generally refers to a technique where a targeted protein is immunoprecipitated from a chromatin preparation to determine the associated DNA sequence and other relevant information (See Latasa et al. 2003 hereby incorporated by reference in its entirety).

As used herein, the term “administering” for in vivo purposes means providing the subject with an effective amount of the DNA-PK inhibitor, siRNA, protein, compound, antibody among others, effective to modulate DNA-PK function of the target cell. Methods of “administering” pharmaceutical compositions are well known to those of skill in the art and include, but are not limited to, microinjection, intravenous or parenteral administration. The compositions are intended for topical, oral, or local administration as well as intravenously, subcutaneously, or intramuscularly. Administration can be effected continuously or intermittently throughout the course of treatment. Methods of determining the most effective means and dosage of administration are well known to those of skill in the art and will vary with the vector used for therapy, the polypeptide or protein used for therapy, the purpose of the therapy, the target cell being treated, and the subject being treated. Single or multiple administrations can be carried out with the dose level and pattern being selected by the treating physician.

### DETAILED DESCRIPTION OF THE INVENTION

Fatty acid synthase (FAS) is a central enzyme in lipogenesis and transcriptionally activated in response to feeding and insulin signaling. The transcription factor USF is required for the activation of FAS transcription, and we show here that USF phosphorylation by DNA-PK, which is dephosphorylated by PP1 in response to feeding, triggers a switch-like mechanism. Under fasting conditions, USF-1 is deacetylated by HDAC9, causing promoter inactivation. In contrast, feeding induces the recruitment of DNAPK to USF-1 and its phosphorylation, which then allows recruitment of P/CAF, resulting in USF-1 acetylation and FAS promoter activation. DNA break/repair components associated with USF induce transient DNA breaks during FAS activation. In DNAPK-deficient SCID mice, feeding-induced USF-1 phosphorylation/acetylation, DNA breaks, and FAS activation leading to lipogenesis are impaired, resulting in decreased triglyceride levels. Thus, the data demonstrates that a kinase central to the DNA damage response mediates metabolic gene activation.

By catalyzing seven reactions in fatty acid synthesis, FAS is a central enzyme in lipogenesis. Regulation of FAS is mainly at the transcriptional level. Applicants have been studying the FAS promoter as a model system to dissect the transcriptional activation by feeding/insulin. We mapped the insulin response sequence (IRS) of the FAS promoter in cultured cells at the −65 E box (Moustaid et al., 1993, 1994), where upstream stimulatory factor (USF)-1/2 heterodimer binds (Moustaid and Sul, 1991; Sawadogo and Roeder, 1985; Wang and Sul, 1995, 1997). Functional analysis and chromatin immunoprecipitation (ChIP) in mice transgenic for various 5′ deletions and mutations of the FAS promoter-CAT reporter gene (Latasa et al., 2000; Moon et al., 2000; Soncini et al., 1995), however, showed that both USF binding to the E box and sterol regulatory element-binding protein-1c (SREBP-1c) binding to the nearby sterol response element (SRE) are required for feeding/insulin-mediated FAS promoter activation in vivo. Furthermore, although increased expression of SREBP-1c (Shimomura et al., 1999), mainly through insulin activation of the PI3K pathway (Engelman et al., 2006; Taniguchi et al., 2006), to bind the FAS promoter is critical for feeding/insulin response, SREBP-1c itself cannot bind its SRE without being recruited by USF, which is constitutively bound to the −65 E box (Griffin et al., 2007; Latasa et al., 2003). Many of the lipogenic promoters contain closely spaced E box and SRE at the proximal promoter region, and we documented a similar mechanism for activation of FAS and mGPAT promoters (Griffin et al., 2007). Thus, USF, along with SREBP-1c, play a critical role in mediating the transcriptional activation of lipogenesis in response to feeding/insulin.

The requirement of USF in induction of lipogenic genes, such as FAS, has been demonstrated in USF-deficient mice (Casado et al., 1999). In humans, SNP studies have implicated USF-1 as a prime candidate of familial combined hyperlipidemia (FCHL) (Pajukanta et al., 2004). Therefore, how does USF regulate lipogenic gene transcription since USF levels do not change during fasting/feeding, and it is constitutively bound to the FAS promoter in both conditions (Wang and Sul, 1995). It is possible that posttranslational modifications of USF underlie its function during fasting/feeding. Insulin regulates metabolism primarily through protein phosphorylation by the well-characterized PI3K cascades (Engelman et al., 2006). Many of the metabolic effects of insulin are also mediated by protein dephosphorylation catalyzed mainly by protein phosphatase-1 (PP1) (Brady and Saltiel, 2001). In this regard, USF has been previously reported to be phosphorylated by various kinases (Cone and Galibert, 2005). However, the significance of USF phosphorylation in lipogenic gene transcription during feeding/insulin is not known. Moreover, USF may not independently function to regulate transcription but recruit coactivators/corepressors. Such recruited factors may also include signaling molecules that transduce extracellular signals to bring about covalent modifications of USF. Thus, it can be postulated that USF and/or its potentially recruited cofactors need to be regulated by dynamic modifications such as phosphorylation/dephosphorylation in response to feeding/insulin.

Here, Applicants show a novel mechanism for the sensing of nutritional/hormonal status by USF to regulate lipogenic gene transcription. Further, Applicants demonstrate that USF-1 phosphorylation by DNA-dependent protein kinase (DNA-PK), which is first dephosphorylated/activated by PP1, is an immediate response to feeding/insulin treatment. Phosphorylation of USF-1 also allows recruitment and acetylation by p300 associated factor (P/CAF). In contrast, during fasting, USF-1 association with histone deacetylase 9 (HDAC9) leads to USF-1 deacetylation. Thus, upon feeding, DNA-PK-deficient SCID mice show impaired USF-1 phosphorylation/acetylation, DNA break, transcriptional activation of the FAS gene, and lipogenesis. The present study shows that DNA-PK is critical for the feeding-dependent activation of lipogenic genes, linking DNA-PK to the insulin-signaling pathway.

Moreover, Applicants recently demonstrated that feeding/insulin activates USF through DNA-PK, a kinase involved in DNA damage repair, and subsequently activates FAS transcription. (See Wong et al., “A Role of DNA-PK for the Metabolic Gene Regulation in Response to Insulin,” Cell 136:1056-1072, Mar. 20, 2009, incorporated herein by reference in its entirety). This insulin signaling pathway involving DNA-PK and USF is first initiated by PP1. Although the molecular mechanism is not well understood, the stimulation of PP1 by insulin has been well documented. For example, insulin inhibits breakdown and promotes synthesis of glycogen primarily by activating PP1. PP1 is known to be compartmentalized in cells by discrete targeting subunits. (Allen et al. 1998). The role of PP1 in transcriptional activation of FAS is to dephosphorylate/activate DNA-PK upon feeding or insulin treatment. USF-1 is then phosphorylated by DNA-PK, allowing recruitment of and acetylation by P/CAF, leading to promoter activation. Further, Applicants demonstrated a requisite role of DNA-PK by employing DNA-PK deficient SCID mice; USF-1 phosphorylation and acetylation is attenuated, blunting transcriptional activation of FAS and de novo lipogenesis in fasting/feeding. (Wong et al. 2009). Thus, Applicants showed DNA-PK is a player in USF regulated transcriptional activation of the FAS gene.

Therefore, USF regulated genes coding for other lipogenic and glycolytic enzymes, such as mitochondrial glycerol-3-phosphate acyltransferase, acetyl-CoA carboxylase and glucokinase, might be possible targets of DNA-PK mediated insulin signaling. Furthermore, in addition to USF, various transcription factors have been reported to regulate a battery of metabolic enzymes (those involved in glycolysis, gluconeogenesis and glycogen and triacylglycerol metabolism) that is regulated during fasting/feeding. Therefore, it is not known if any additional transcription factors aside from USF, if any, are phosphorylated by DNA-PK in response to feeding/insulin. In addition to phosphorylating transcription factor(s), it is contemplated that DNA-PK might also play a role in regulating enzymes that are under control of feeding/insulin. In this regard, as an insulin signaling molecule, DNA-PK might potentially phosphorylate proteins including kinases that are activated by insulin. Last but not least, with DNA-PK's role as an insulin signaling molecule in activating lipogenesis, DNA-PK might serve as a pharmacological target for obesity and diabetes treatment. Thus, identification of DNA-PK as a signaling molecule in activating lipogenic genes by insulin has brought us a step closer to understanding how cells respond to insulin.

### DISCUSSION

FAS levels in the liver change drastically during varying nutritional states, correlating with circulating insulin/glucagon levels. During fasting, fatty acid synthesis is virtually absent. However, upon feeding, accompanying insulin secretion, fatty acid synthesis is induced drastically. While many metabolic effects of insulin are mediated through protein phosphorylation by the activation of the well-characterized PI3K cascade, insulin can also exert metabolic effects through dephosphorylation catalyzed mainly by PP1. A central issue in metabolic regulation is to define coordinated molecular strategies that underlie the transition from fasting to feeding, such as the transcriptional activation of lipogenesis along specific transduction pathways. Here, Applicants report a novel pathway that underlies the feeding/insulin response, which is based on posttranslational modifications of a key transcription factor, USF-1, by an atypical kinase, DNA-PK.

### Differential Binding of USF-1-Interacting Proteins to Lipogenic Gene Promoters in Fasted and Fed States

The results show that USF recruits three different coregulator classes to lipogenic gene promoters. They are (1) the DNA break/repair machinery, (2) kinase/phosphatase, and (3) HAT/HDAC family. The distinct binding pattern of USF-interacting proteins on the FAS promoter in response to feeding/fasting is correlated with lipogenic gene activation/repression, which involve molecular events that require the presence of specific coactivators/corepressors, respectively.

FAS and other lipogenic enzymes such as mGPAT are coordinately regulated by feeding/insulin involving USF and SREBP-1c binding to the closely spaced E box and SRE, respectively. We show here that the USF-1 bound to the −65 E box recruits various USF-1-interacting proteins as well as SREBP-1c to bind SRE. Herein, we address the molecular function of various USF-1-interacting proteins and USF-1 modifications required for FAS promoter activation. Furthermore, FAS and mGPAT have the same differential recruitment of distinct USF-interacting proteins, indicating a common key mechanism in the induction of lipogenic gene transcription in response to fasting/feeding.

### Phosphorylation-Dependent Acetylation of USF-1 Functions as a Sensor for Nutritional Status

Because USF-1 levels and its binding to the E box are unaltered between fasting/feeding, it can be predicted that USF-1 is regulated posttranslationally. Even though the changes in phosphorylation states of metabolic enzymes during the transition between fasting/feeding are common and well understood, the posttranslational modifications of transcription factors in these metabolic states are not well studied. We show here that S262 and the nearby K237 of USF-1 are modified in response to fasting/feeding. The S262 of USF-1 as well as nearby residues are conserved among mammalian species but are not found in USF-2 even though there is a 44% overall homology between USF-1 and USF-2 (Cone and Galibert, 2005). Activation of the FAS gene by feeding has been shown to be impaired by 80% in either USF-1 or USF-2 knockout mice (Casado et al., 1999). Thus, USF functions as a heterodimer, and both USF-1 and USF-2 were found to bind the FAS promoter (Wang and Sul, 1995, 1997). However, the unique S262 of USF-1 points toward its pivotal role as a sensor for lipogenic gene transcription. There is increasing evidence for acetylation of some transcription factors in addition to the well-recognized histone acetylation (Gu and Roeder, 1997), and reversible acetylation may be critical in regulation of transcription factor activity in response to different stimuli. However, USF acetylation has never been reported. Here, we have addressed USF-1 as a primary substrate for HAT/HDAC. The functional significance of acetylation of transcription factors appears to be varied. In the case of p53, acetylation results in stimulation of DNA binding, whereas acetylation of E2F may change protein stability (Martinez-Balbas et al., 2000). The fact that USF levels do not change during fasting/feeding and that USF acetylation does not affect DNA binding but affects FAS promoter activation suggests transactivation results from USF acetylation, and our study demonstrates that acetylation of USF-1 at K237 increases FAS promoter activity. Further studies are needed to clarify the exact functional consequence of USF acetylation. Deacetylation is mainly mediated by HDACs that generally function as transcriptional repressors. HDAC9 is recruited to the FAS promoter in the fasted state to deacetylate USF-1. Although HDAC9 has been shown to associate with transcription factors to repress transcription (Mejat et al., 2005), to our knowledge, HDAC9 deacetylation of USF-1 that Applicants report here is the first nonhistone substrate of HDAC9.

Crosstalk between acetylation and phosphorylation is well recognized. In the present study, K237 acetylation is dependent on S262 phosphorylation in response to feeding/insulin by preferential interaction with P/CAF rather than HDAC9. Thus, the phosphorylation-dependent acetylation of USF-1 functions as a dynamic molecular switch in sensing the nutritional transition from fasting to feeding. Such a multistep switch provides a way to fine-tune transcription of lipogenic genes in response to different nutritional states.

### PP1-Mediated Dephosphorylation of DNA-PK is Critical for Feeding-Dependent Lipogenic Gene Transcription

It has been well established that PI3K pathway mainly mediates insulin signaling for metabolic regulation (Engelman et al., 2006). Our in vitro phosphorylation studies and the fact that S262 phosphorylation is abolished in DNA-PK-deficient mice point to the notion that DNA-PK is the kinase for the S262 phosphorylation occurring in the fed condition. However, DNA-PK is not known to be a component in the PI3K pathway or in the insulin-signaling pathway. Although DNA-PK was previously implicated in phosphorylation of 5473 of PKB/Akt (Feng et al., 2004), recent research indicates that mTORC2, another member of PIKK, is the authentic kinase that phosphorylates this critical site of PKB/Akt (Sarbassov et al., 2005). However, our present study shows a link between DNA-PK and insulin-signaling pathway.

Although the molecular mechanism is complex, the stimulation of PP1 by insulin has been well documented. For example, insulin inhibits breakdown and promotes synthesis of glycogen by activating primarily PP1. PP1 is compartmentalized in cells by discrete targeting subunits, and several proteins called “protein targeting to glycogen (PTG) can target PP1 to the glycogen particle where PP1 dephosphorylates enzymes in glycogen metabolism (Printen et al., 1997). Recent studies indicate that PP1 can rapidly move between subcellular compartments with the aid of targeting units. PNUT, a PP1 associated cofactor, may act as a nuclear targeting subunit of PP1 (Allen et al., 1998). We postulate that feeding/insulin might regulate PNUT-mediated nuclear translocation of PP1 into the nucleus to activate DNA-PK. Thus, PP1-mediated dephosphorylation of DNA-PK is critical in transmitting the feeding/insulin signal to regulate lipogenic genes.

Among USF-interacting proteins, DNA-PK, along with Ku70, Ku80, PARD-1, and TopoIIβ, are identified. These proteins are known to function in double-strand DNA break/repair, and it has recently been shown that a transient double-strand DNA break is required for estrogen receptor-dependent transcription. Although Ku70, Ku80, and DNA-PK are in the same complex with PARP-1 and TopoIIβ, their function in DNA break for transcriptional activation has not been reported. Here, we identified all components of DNA break/repair machinery for transcriptional activation of the FAS promoter by fasting/feeding, and we observed transient DNA breaks that preceded transcriptional activation.

We show here a unique function of DNA-PK as a signaling molecule in response to feeding/insulin. DNA-PK is required for USF-1 complex assembly and recruitment of its interacting proteins. Therefore, DNA-PK-mediated USF-1 phosphorylation governs interaction between USF-1 and its partners. SREBP-1 interacts more efficiently with the phosphorylated USF-1, which, in turn, enhances the interaction between USF-1 and DNA-PK, leading to USF-1 phosphorylation, an indication of positive feed-forward regulation. Thus, impaired transcriptional activation of lipogenic genes in DNA-PK-deficient SCID mice is probably due to the dual effects of DNA-PK on USF-1 phosphorylation for feeding/insulin signaling and the transient DNA breaks required for transcriptional activation. In SCID mice, the absence of the feeding-induced transient DNA breaks in the FAS promoter could be attributed to the impairment of feeding/insulin-induced USF phosphorylation by DNA-PK, which results in a failure to recruit various USF-1-interacting proteins, including those for transient DNA breaks such as TopoIIβ.

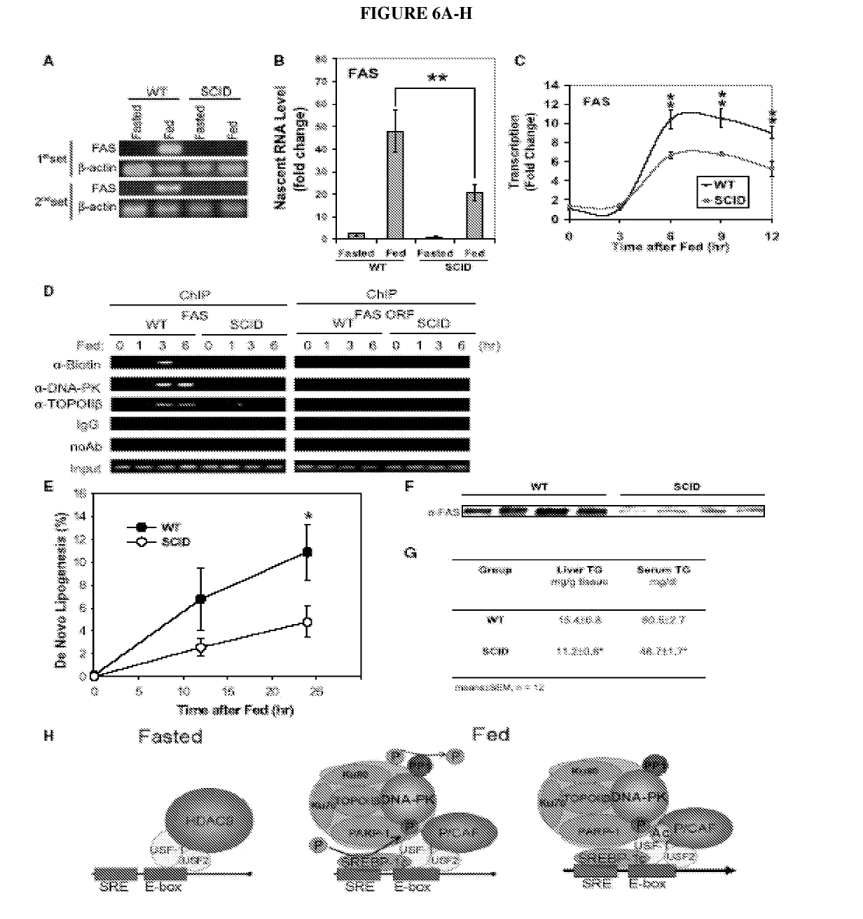

Taken together, we propose the following model for the mechanism underlying USF function in the transcriptional regulation of lipogenic genes during fasting/feeding (FIG. 6H). In the fasted state, USF-1 recruits HDAC9, which deacetylates USF-1 to repress transcription despite its binding to the E box (FIG. 6H, left panel). Upon feeding, DNA-PK, which is dephosphorylated/activated by PP1, phosphorylates USF-1, which then recruits SREBP-1 and other USF-1-interacting proteins. Thus, DNA-PK-catalyzed phosphorylation of USF-1 allows P/CAF recruitment and subsequent acetylation of USF-1 (FIG. 6H, right panel). As a result, FAS transcription is activated by USF-1 in a reversible manner in response to nutritional status.

### EXPERIMENTAL

The following examples serve to illustrate certain preferred embodiments and aspects of the present invention and are not to be construed as limiting the scope thereof.

### Experimental Procedures

### Purification of USF-1-Interacting Proteins and Preparation of Nuclear Extracts

TAP was performed as described previously (Griffin et al., 2007). Purified protein mixture was subjected to mass spectrometry. Liver nuclear extracts were prepared by centrifugation through sucrose cushion in the presence of NaF.

### Chromatin Immunoprecipitation

Livers from fasted or fed mice were fixed with DSG at 2 mM for 45 min at RT before formaldehyde crosslinking. ChIP was performed as described previously (Latasa et al., 2003).

### In Vitro Phosphorylation, Acetylation, and DNA-PK Kinase Assay

In vitro phosphorylation and acetylation were performed using recombinant/purified enzymes. DNA-PK kinase assay was performed with nuclear extracts pretreated with or without wortmannin using SignaTect DNA-PK assay system (Promega) and g32P-ATP (Roche).

### Nuclear Run-On Assay and Preparation of Nascent RNA

Nuclei were isolated as described previously (Paulauskis and Sul, 1989) for nascent RNA and nuclear run-on assay (See the Supplemental Experimental Procedures for further details).

### Immunoprecipitation, GST Pull-Down, Luciferase Reporter Assays

Immunoprecipitation from nuclear extracts was performed under standard procedures. GST pull-down was performed as described previously (Griffin et al., 2007). Luciferase assays were performed in 293FT cells using Dual-Luc reagent (Promega).

### RT-PCR Analysis

RNA was isolated and reverse transcribed for PCR or qPCR.

### Measurement for Metabolite and Hormone Levels

Insulin, glucose, NEFA, and triglycerides were measured by ELISA (Crystal), glucometer (Roche), NEFA C kit (Wako), and Infinity kit (Thermo), respectively.

### De Novo Lipogenesis (DNL)

Fatty acids formed during a 4 hr ²H₂O body water labeling (see Supplemental Experimental Procedures for further details).

### Statistical Analysis

The data are expressed as the means±SE of the means. Student's t test was used (*p<0.05, **p<0.01, ***p<0.005, and ****p<0.0001).

### Supplemental Experimental Procedures

### Antibodies, Animals, Cell Culture, and Transfection

Rabbit polyclonal antibodies were raised against peptides corresponding to aa 252-265 (QELRQSNHRL(S)EEL) [SEQ ID. NO. 43] and 231-244 (CSMEST(K)SGQSKGG) [SEQ ID. NO. 44] of USF-1. SCID in C57BL/6J background and wild type The following commercially available antibodies were used: Monoclonal Anti-USF-1 (M01,M02) (Abnova), M2 anti-FLAG (Sigma), anti-DNA-PK (4F1005) (Upstate), anti-AcK (4G12) (Upstate), antiphosphoserine (Calbiochem), anti-S/TQ ATM/ATR substrate (Cell Signaling), anti-PAR (Alexis Biochemical), anti-HA (Covance) and polyclonal anti-USF-1 (C-20), anti-Actin, anti-Biotin, anti-p300, normal IgG, anti-HDAC9, anti-GAPDH, anti-PARP-1, anti-Ku70, anti-Ku80, anti-TopoIIβ anti-PP1, anti-P/CAF, anti-FAS (Santa Cruz) and anti-p53 (Santa Cruz).

C57BL/6J male mice (Jackson laboratory) were used at 7 wks of age unless specified. For fasting/feeding experiments, mice were fasted for 40 hrs and then fed a high carbohydrate, fat-free diet for indicated time periods. HepG2 cells were grown in DMEM supplemented with 10% fetal bovine serum and 100 units/ml penicillin/streptomycin. M059J and M059K were from ATCC and grown in the same medium containing 4 mg/ml glutamine. HepG2 cells were maintained in serum free media overnight prior to insulin treatment. For insulin treatment, HepG2 cells were treated with 100 nM insulin or DMSO for 30 min. 293FT cells in DMEM supplemented with 10% fetal bovine serum and 100 units/ml penicillin/streptomycin/neomycin or 293F cells in 293 Freestyle medium were transfected with expression constructs or siRNA (Santa Cruz) using lipofectamine 2000 (Invitrogen) or 293 Fectin (Invitrogen), respectively. 293 cells were treated with either control DMSO or OA at 1 uM and Taut at indicated concentrations for 2 hrs. Expression vectors for DNA-PK and mutants, HAT, HDAC9, PP1, Kus and −0.7 p53-Luc were from laboratories of Drs. Meek, Kouzarides, Zelent, Lamond, Shay and Oren respectively. siRNA for knockdown of DNA-PK is commercially available at Santa Cruz Biotechnology Inc.

### Purification of USF-1-Interacting Proteins and Preparation of Nuclear Extracts

The 293F cells were transfected with USF-1-FLAG-TAP or empty TAP vector. Briefly, nuclear extracts were subjected to two-step affinity purification using calmodulin and streptavidin resins (Stratagene) (Griffin et al., 2007). Purified proteins were concentrated by Centricon YM-3 (Amicon) and analyzed by SDS-PAGE, followed by silver staining (Invitrogen). Purified protein mixture was subjected to 2D “MudPIT” Run (cation exchange/RP LC-MS/MS) using a Finnigan LCQ Deca XP mass spectrometer in NanoLC/ESI mode. Sequest program was used for interpretation of the mass spectra. For USF-1 interaction experiments, nuclear extracts were added to immobilized GST-USF-1-FLAG fusion protein and incubated overnight. After extensive washing, bound proteins were eluted with glutathione and then subjected to a second round of purification on anti-FLAG resins (Sigma). Eluted complexes were neutralized with glycine and subjected to MS analysis.

For liver nuclear extracts, mice were fasted for 40 hrs and then fed a high carbohydrate, fat-free diet for 16 hrs or indicated time periods. Nuclear extracts were prepared by centrifugation through sucrose cushion in the presence of NaF. (Griffin et al., 2007). For 293 cells, nuclear extracts were prepared by high salt extraction (Andrews and Faller 1991).

### Chromatin Immunoprecipitation

Livers from fasted or fed mice were fixed with DSG at 2 mM for 45 min at RT before formaldehyde cross-linking Soluble chromatin was quantified by absorbance at 260 nm, and equivalent amounts of input DNA were immunoprecipitated. ChIP was performed as described previously (Latasa et al., 2003). For detection of DNA-break, DNA-breaks were labeled with Biotin-16-dUTP (Roche), and chromatin was subjected to ChIP using anti-biotin antibodies (Ju et al., 2006). For real time PCR of ChIP samples, the fold enrichment values were normalized to the control IgG.

### In Vitro Phosphorylation, Acetylation, and DNA-PK Kinase Assay

In vitro phosphorylation reactions were performed using DNA-PK (Promega), PKA (Upstate), PKC (Upstate) and ATP (Promega). Wortmannin was used at 2 uM for in vitro phosphorylation. For in vitro acetylation, proteins were incubated with P/CAF (Upstate) using acetyl CoA (Sigma) as the donor of the acetyl group. DNA-PK kinase assay was performed using mouse nuclear extracts pretreated with or without wortmannin (2 uM) using SignaTect DNA-PK assay system (Promega) and γ³²P-ATP (Roche).

### Nuclear Run-On Assay and Preparation of Nascent RNA

Nuclei from livers of 3-5 mice were isolated by centrifugation through sucrose cushion as described previously (Paulauskis and Sul, 1989). For nascent RNA measurement, nuclei were treated with DNase (Roche) and purified using RNeasy kit (Qiagen). For nuclear run-on assay, nuclei were either incubated with biotin UTP (Roche) or UTP (Sigma) in in vitro transcription buffer. Labeled RNA purified using RNeasy kit (Qiagen) were pulled down using avidin beads (Sigma) before RT-qPCR (Patrone et al., 2000).

### Immunoprecipitation, GST Pull-Down, Luciferase Reporter Assays

For immunoprecipitation, nuclear extracts were incubated with the specific antibodies overnight at 4° C. followed by incubation with protein G agarose beads (Santa Cruz), washed and separated by SDS-PAGE. Proteins were transferred onto nitrocellulose membranes (Bio-Rad) and Western blotting was performed. For GST pulldown, bacterially expressed GST proteins were first incubated with glutathione-agarose (Santa Cruz) followed by incubation with ³⁵S labeled proteins, and autoradiography was performed (Griffin et al., 2007). Plasmids containing full length cDNA of PP1, PARP-1 and TopoIIβ (Open Biosystem) were used for in vitro translation. Purified recombinant PARP-1 (Alexis) and PP1 (New England Biolabs) were used in the GST-pull down assay. The 293FT cells were transfected with −444-FAS-Luc along with various expression constructs and siRNA (Santa Cruz) using Lipofectamine 2000 reagent (Invitrogen), and luciferase assays were performed using Dual-Luc reagent (Promega).

### RT-PCR Analysis

Four mg of total RNA isolated using Trizol reagent (Gibco BRL) were reverse transcribed and the resultant cDNAs were amplified by semi-quantitative PCR or real time qPCR. For Real-time RT-qPCR, the relative mRNA levels of gene markers were quantified with β-actin as the internal control using EVA dye (Biochain) as the probe. Statistical analysis of the qPCR was obtained using the (2^{-ΔΔCt}) method.

### Measurements for Metabolite and Hormone Levels

Insulin levels were measured by an insulin ELISA kit (Crystal). Whole blood glucose concentration was measured with ACCU-CHEK (Roche) glucometer. Serum NEFAs levels were measured by NEFA C kit (Wako). Serum triglyceride levels and liver triglyceride levels after extraction by Folch method were measured by Infinity Triglyceride Kit (Thermo).

### Measurement of De Novo Lipogenesis (DNL)

Fatty acids synthesized during a 4 hrs ²H₂O body water labeling were measured as described previously (Turner et al., 2003). Mass isotopomer distribution analysis (MIDA) was used. Fractional DNL contribution was calculated as previously described by f_{DNL}=M1_{FA}/A₁∞_{FA}.

### Blue Native-PAGE for Detection of USF-1 Complex

For detection of USF-1 complex, TAP eluates were incubated with the BN-PAGE loading dye at 4° C. for 30 min, the samples were loaded onto a 6% BN gel and subjected to PAGE. After electrophoresis, nitrocellulose membrane was destained with methanol before Western blotting with anti-FLAG antibodies. USF-1-TAP eluates were incubated at 4° C. for 30 min with 2 μg of antibodies (anti-GAPDH or anti-USF-1) for supershifting (Schagger et al., 1994).

### Primer Sequences

Gene specific target sequences were as follows: The primer pairs used in semiquantitative RT-PCR were GAPDH (sense—CATCACCATCTTCCAGGAGCG (SEQ ID. NO. 1); antisense—TGACCTTGCCCACAGCCTTG (SEQ ID. NO. 2)); DNA-PK (sense—GCC AAA GCG CAT TGT TAT TCG (SEQ ID. NO. 3); antisense—GGG GTC ACT GTT ATT AGC CAC (SEQ ID. NO. 4)); Ku70 (sense—TCC TGC AGC AGC ACT TCC GCA (SEQ ID. NO. 5); antisense—CAG TGT AGG TAC AGT GAG CTT (SEQ ID. NO. 6)); Ku80 (sense—GCT TTC CGG GAG GAG GCC ATT (SEQ ID. NO. 7); antisense—CTC TTG GAT TCC CCA CAC ATC (SEQ ID. NO. 8)); PARP-1 (sense—CTG CAC CAG ACA CCA CAA AAC (SEQ ID. NO. 9); antisense-TTC CCT GGG GAA GCC AGT AAG (SEQ ID. NO. 10)); P/CAF (sense—AGA GGT AGT GTG CTT GAA GGA (SEQ ID. NO. 11); antisense CTC TTT AAG GAT GTC TAC CCA (SEQ ID. NO. 12)); PP1α (sense—TGG ATG AGA CCC TCA TGT GTT (SEQ ID. NO. 13); antisense—TGG GAG ATT AGA TGC TGC TAT (SEQ ID. NO. 14)); PP1γ (sense—GCA CGC CCT GGG GAT GAG GTG (SEQ ID. NO. 15); antisense—CGC AGA ATA AAG AAT GTA GCC (SEQ ID. NO. 16)); Topoisomerase IIβ (sense—GTA AAG GCC GAG GGG CAA AGA (SEQ ID. NO. 17); antisense—AAT GTT CGT GCT CTT TGG GCA (SEQ ID. NO. 18)).

The primer pairs used in quantitative RT-PCR were FAS (sense-TGCTCCCAGCTGCAGGC (SEQ ID. NO. 19); antisense-GCCCGGTAGCTCTGGGTGTA (SEQ ID. NO. 20)), mGPAT (sense—CTG CTA GAA GCC TAC AGC TCT (SEQ ID. NO. 21); antisense—CAG CAC CAC AAA ACT CAG AAT (SEQ ID. NO. 22)), p53 (sense—AAA GGA TGC CCA TGC TAC AGA GGA (SEQ ID. NO. 23); antisense—AGT AGA CTG GCC CTT CTT GGT CTT (SEQ ID. NO. 24)), β-actin (sense-GACCGAGCGTGGCTACAGCTTCA (SEQ ID. NO. 25); antisense-CCGTCAGGCAGCTCATAGCTCT (SEQ ID. NO. 26)).

Primer sequences for amplification of the proximal region of the mouse mitochondrial GPAT promoter were 5′-ACAGCCACACTCACAGAGAATGGGGC-3′ (SEQ ID. NO. 27) and 5′-GAAGAGGCAGACTCGGCGTTCCGGAG-3′ (SEQ ID. NO. 28). Primer sequences for amplification of the proximal region of the mouse p53 promoter were 5′-GTT ATG GCG ACT ATC CAG CTT-3′ (SEQ ID. NO. 29) and 5′-CCC CTA ACT GTA GTC GCT ACC-3′ (SEQ ID. NO. 30). Primer sequences for amplification of the proximal region of the human FAS promoter were 5′-GCA CAC GTG GCC CCG GCG GAC-3′ (SEQ ID. NO. 31) and 5′-CAC GCC ACA TGG GCT GAC AGC-3′ (SEQ ID. NO. 32). Primer sequences for amplification of the proximal region of the FAS-Luc promoter were 5′-CAG CCC CGA CGC TCA TTG G-3′ (SEQ ID. NO. 33) and 5′-CTT CAT AGC CTT ATG CAG TTG-3′ (SEQ ID. NO. 34). Primer sequences for amplification of the proximal region of the p53-Luc promoter were 5′-GAC TTT TCA CAA AGC GTT CCT-3′ (SEQ ID. NO. 35) and 5′-AGC CAG

GGT GAG CAC GTG GGA-3′ (SEQ ID. NO. 36). Primers used for real time PCR were identical to those used in determination of nascent RNA from mouse liver and they were β-actin (Sense: GTGGCATCCATGAAACTACAT (SEQ ID. NO. 37); antisense: GAGCCAGAGCAGTAATCTCCT (SEQ ID. NO. 38)); FAS (sense: ACGTGACACTGCTGCGTGCCA (SEQ ID. NO. 39); antisense: ATACTCAGGTGTCATTCTGTG (SEQ ID. NO. 40)).

### Example I

### Results

Identification of USF-Interacting Proteins and their Occupancy on Lipogenic Gene Promoters During Fasting/Feeding

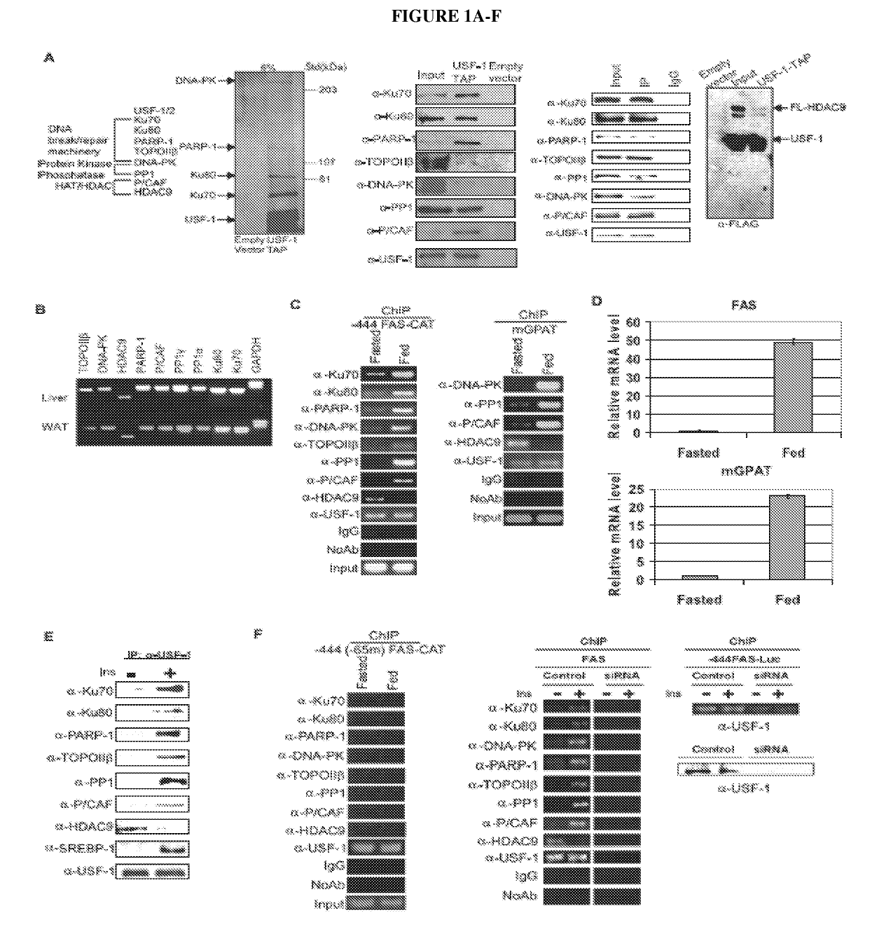

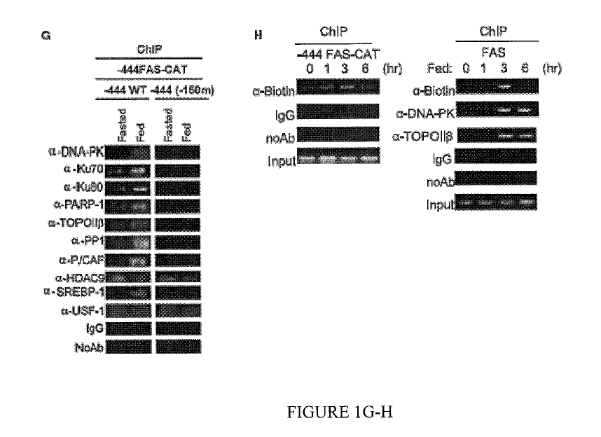

It was previously shown that USF is required for the regulation of FAS promoter activity in fasting/feeding (Wang and Sul, 1995, 1997). However, USF is constitutively bound to the FAS promoter (Griffin et al., 2007; Latasa et al., 2003). It was postulated that USF may repress or activate the FAS promoter by recruiting distinct cofactors in fasted and fed conditions. Therefore tandem affinity purification (TAP) and mass spectrometry (MS) analysis was performed. The USF-interacting proteins were purified from nuclear extracts prepared from 293 cells overexpressing USF-1 tagged with streptavidin and calmodulin-binding peptides (TAP tagged) as well as a FLAG epitope at its carboxyl terminus. In addition to USF-1 and USF-2, we identified seven polypeptides in the eluates by MS analysis (FIG. 1A, left panel and Table S2). These proteins fall into three categories: (1) DNA break/repair components DNA-PK and its regulatory subunits, Ku70, Ku80, as well as poly(ADP-ribose) polymerase-1 (PARP-1) and Topoisomerase IIβ (TopoIIβ), (2) protein phosphatase PP1, and (3) P/CAF, which belongs to the histone acetyltransferases (HAT) family. Interestingly, we detected some of the USF-interacting proteins to be poly(ADP-ribosyl)ated (FIG. 12E). TAP using cells that were first crosslinked by DSP showed identical USF-1-interacting proteins (data not shown). Referring to Table S2 (below), the peptides of USF-1 interacting proteins are identified by MS.

TABLE S2

| Protein | Peptide sequence | |
|---|---|---|
| USF-1 | K.YVFRTENGGQVMYR.V2 SEQ ID. NO. 45 | K.ACDYIQELR.Q2 SEQ ID. NO. 46 |
| | R.QQVEDLKNKNLLLR.A2 SEQ ID. NO. 47 | K.YVFRTENGGQVMR.V2 SEQ ID. 45 |
| | K.YVFRTENGGQVVYR.V2 SEQ ID. NO. 45 | R.TENGGQVMYR.V2 SEQ ID. NO. 48 |
| | R.THPYSPKSEAPR.T2 SEQ ID. NO. 49 | K.ACDYIQELR.Q1 SEQ ID. NO. 50 |
| | K.ACDYIGELR.Q2 SEQ ID. NO. 46 | R.LSEELQGLDQLDNDVLR.Q2 SEQ ID. NO. 46 |
| | R.LSEELQGLDQLQLDNDVLRQQVEDLKNK.N3 | R.QQVEDLKNK.2 SEQ ID. NO. 52 |
| | SEQ. ID. NO. 51 | |
| | R.QQVEDLKNKNLLLR.A SEQ ID. NO. 53 | |
| USF-2 | R.RDKINNWIVQLSK.I2 SEQ ID. NO. 54 | R.DKINNVIVQLSK.I2 SEQ ID. NO. 55 |
| | R.DNINNVVIVQLSK.I2 SEQ ID. NO. 55 | K.INNWIVQLSK.I1 SEQ ID. NO. 56 |
| | K.INNWIVQLSK.I SEQ ID. NO. 57 | |
| Ku70 | R.ILELDQFKGQQGQKR.P2 SEQ ID. NO. 58 | R.IMLFTNEDNPHGNDSAK.A2 SEQ ID. NO. 59 |
| | K.AGDLRDTGIFLDLMHLK.K2 SEQ ID. NO. 60 | K.TRTFNTSTGGLLLPSDTKR.S3 SEQ ID. NO. 61 |
| | K.TRTFNTSTGGLLLPSDTKR.S2 SEQ ID. NO. 61 | R.TFNTSTGGLLLPSDTKR.S2 SEQ ID. NO. 62 |
| | K.CLEKEVAACR.Y2 SEQ ID. NO. 63 | R.NLEALALDLMEPEQAVDLTLPK.V3 SEQ ID. NO. 64 |
| | R.NEALALDLMEPEQAVDLTLPKVEAMNK.R3 | R.LGSLVDEFKELVYPPDYNPEGK.V2 SEQ ID. NO. 66 |
| | SEQ ID. NO. 65 | |
| | R.NLEALALDLMEPEQAVDLTLPKVEAMNKR.L3 | K.GTLGKFTVPMILK.E2 SEQ ID. NO. 68 |
| | SEQ ID. NO. 67 | |
| | K.SGLKKQELLEALTK.H SEQ ID. NO. 69 | |
| Ku80 | R.HLMLPDFDLLEDIESK.I1 SEQ ID. NO. 70 | K.KYAPTEAQLNAVDALIDSMSLAK.K2 SEQ ID. NO. 71 |
| | K.YAPTEAQLNAVDALDSMSLAK.K2 | R.LFQCLLHR.A2 SEQ ID. NO. 73 |
| | SEQ ID. NO. 72 | |
| | K.IKTLFPLIEAK.K2 SEQ ID. NO. 74 | K.ASFEESNQLINHIEQFLDTNETPYFMK.S |
| | | SEQ ID. NO. 75 |
| PARP-1 | K.CSESIPKDSLR.M2 SEQ ID. NO. 76 | K.TEAAGGVTGKGQDGIGSKAEK.T2 SEQ ID. NO. 77 |
| | K.RKGDEVDGVDEVAK.K2 SEQ ID. NO. 78 | K.VCSTNDLKELLFNK.Q2 SEQ ID. NO. 79 |
| | R.VVSEDFLQDVSASTK.S2 SEQ ID. NO. 80 | K.SKLPKPVQDLIK.M2 SEQ ID. NO. 81 |
| | K.KPPLLNNADSVQAK.V SEQ ID. NO. 82 | |
| TOPOIIβ | K.GIPVVEHKVEK.V2 SEQ ID. NO. 83 | R.RLHBLPEQFLYGTATK.H2 SEQ ID. NO. 84 |
| | R.LHGLPEQFLYGTATK.H2 SEQ ID. NO. 85 | |
| DNA-PK | R.CGAALAGHQIR.G 2 SEQ ID. NO. 86 | R.ICSKPVVLPK.G2 SEQ ID. NO. 87 |
| | R.LYSLALHPNAFKR.L2 SEQ ID. NO. 88 | K.WLLAHCGRPQTECR.H2 SEQ ID. NO. 89 |
| | R.FNNYVDCMKK.F2 SEQ ID. NO. 90 | K.INQVFHGSCITEGNELTK.T2 SEQ ID. NO. 91 |
| | R.SSFDWLTGSSTDPLVDHTSPSSDSLLFAHK.R3 | R.SSFDWLTGSSTDPLVDHTSPSSDSLLFAHKR.S3 |
| | SEQ ID. NO. 92 | SEQ ID. NO. 93 |
| | R.LGLPGDEVDNKVK.G2 SEQ ID. NO. 94 | R.LLQIIERYPEETLSLMTK.E2 SEQ ID. NO. 95 |
| | K.GANRTETVTSFR.K 2 SEQ ID. NO. 96 | K.KGGSWIQEINVAEKNWYPR.Q3 SEQ ID. NO. 97 |
| | K.KGGSWIQEINVAEKNWYPR.Q2 SEQ ID. NO. 97 | |
| PP1 | K.NVQLQENEIR.G2 SEQ ID. NO. 98 | K.IKYPENFFLLR.G2 SEQ ID. NO. 99 |
| | K.IFCCHGGLSPDLQSMEQIRR.I2 SEQ ID. NO. 100 | K.IFCCHGGLSPDLQSMEQIRR.I3 SEQ ID. NO. 100 |
| | K.TFTDCFNCLPIAAIVDEK.I2 SEQ ID. NO. 101 | K.YGQFSGLNPGGRPITPPR.N2 SEQ ID. NO. 102 |
| | K.TFTDCFNCLPIAAIVDEK.J SEQ ID. NO. 101 | K.YGQFSGLNPGGROITOOR.N SEQ ID. NO. 103 |
| PICAF | K.MTDSHVLEEAKKPR.V2 SEQ ID. NO. 104 | K.MTDSHVLEEAK#KPR.V2 SEQ ID. NO. 104 |
| | K.HDILNFLTYADEYAIGYFK.K2 SEQ ID. NO. 105 | K.HDILNFLTYADEYAIGYFKK.Q2 SEQ ID. NO. 106 |
| | K.YVGYIKDYEGATLMGCELNPR.I2 SEQ ID. NO. 107 | K.SK#EPRDPDQLYSTLK.S2 SEQ ID. NO. 108 |
| | K.SHQSAWPFMEPVKR.T2 SEQ ID. NO. 109 | K.SHQSAWPFMEPVKR.T3 SEQ ID. NO. 109 |
| | R.VFTNCKEYNPPESEYYK.C2 SEQ ID. NO. 110 | |
| HDAC9 | K.QLQCELLLIQQQQQIQK.Q2 SEQ ID. NO. 111 | |

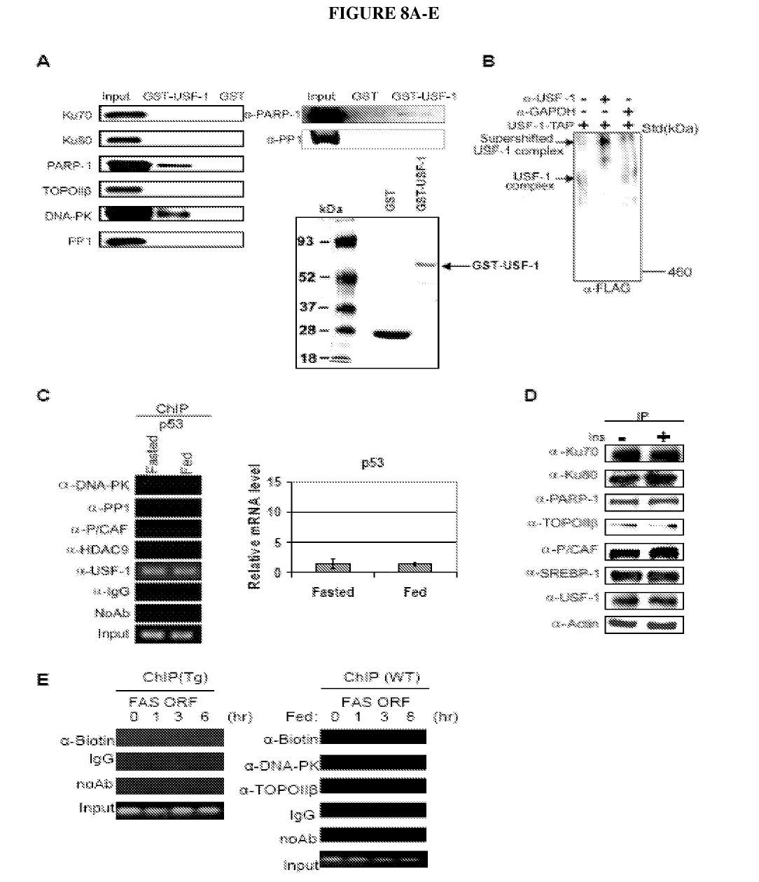

Applicants detected at least five of the polypeptides having molecular weights corresponding to the above identified proteins by silver staining of the TAP eluates separated by SDS-PAGE (FIG. 1A, second left panel). Blue native (BN) gel electrophoresis of the TAP eluates revealed the presence of a large USF-1-containing complex (FIG. 8B). Immunoblotting of the eluates using antibodies against each of the seven polypeptides further confirmed the presence of all seven polypeptides that were copurified with TAP-tagged USF-1 (FIG. 1A, third left panel). These identified proteins were specific to USF-1 because none of them were found with the control TAP tag. Confirming USF-1 interaction, coimmunoprecipitation followed by immunoblotting revealed the presence of all interacting proteins in endogenous USF-1 immunoprecipitates (FIG. 1A, second right panel). Furthermore, GST pull-down assay showed that DNA-PK and PARP-1, but not TopoIIβ, Ku70/Ku80, and PP1, can directly interact with USF-1 (FIG. 8A).

Applicants also attempted to purify and identify USF-interacting proteins by incubating liver nuclear extracts with bacterially expressed TAP-tagged USF immobilized on agarose beads. MS analysis identified an additional USF-interacting protein HDAC9, a transcriptional corepressor that belongs to the class II HDAC family, which was copurified with USF-1 when the nuclear extracts from fasted mice were used (data not shown). The interaction between HDAC9 and USF-1 was confirmed by detection of HDAC9 copurified with USF-1 by TAP in cells overexpressing HDAC9 and USF-1 (FIG. 1A, right panel). Overall, except for P/CAF, which has been implicated to function with USF for histone modification in chromosomal silencing (West et al., 2004), none of the above proteins have previously been shown to interact with USF.

All of the USF-interacting proteins were expressed in lipogenic tissues, liver, and white adipose tissue (WAT) (FIG. 1B). Applicants next performed ChIP in livers of fasted and fed transgenic mice expressing a CAT reporter gene driven by the −444 FAS promoter, a minimal FAS promoter sufficient for full response to fasting/feeding and diabetes/insulin treatments (Latasa et al., 2000, 2003; Moon et al., 2000). As shown before, binding of USF in both fasted and fed conditions was detected (FIG. 1C, left panel). In the fasted state, however, Applicants detected the corepressor HDAC9 bound to the FAS promoter, but not other interacting proteins that we identified by TAP-MS. Upon feeding, HDAC9 was no longer bound to the promoter, but the FAS promoter was now occupied by the coactivator P/CAF, DNA break/repair components that include DNA-PK, Ku70/80, PARP-1, TopoIIβ, as well as PP1 (FIG. 1C, left panel). ChIP analysis of the mGPAT promoter was also performed using antibodies against proteins that represent each of the three categories of the USF-interacting proteins. Similar to what we observed with the FAS promoter, USF-1 was bound to the mGPAT promoter in both fasted and fed conditions (FIG. 1C, right panel). Furthermore, as seen with the FAS promoter, HDAC9 was bound to the mGPAT promoter only in fasting, whereas DNA-PK, PPI, and P/CAF were bound only in the fed state. The regulated expression of FAS and mGPAT was also verified in these mice. As predicted, FAS and mGPAT mRNA levels were very low in livers of fasted mice, but upon feeding, they were induced drastically to ˜50- and 25-fold, respectively (FIG. 1D). The similar binding pattern of USF-interacting proteins suggests a common mechanism for lipogenic induction involving USF and its interacting proteins in response to feeding. Overall, USF-1 is constitutively bound to the FAS and other lipogenic promoters in both metabolic states, whereas USF-interacting proteins are bound in a fasting/feeding-dependent manner. Applicants next investigated whether this is due to the differential interaction of USF with these proteins by employing insulin-responsive HepG2 cells overexpressing USF-1. The levels of various USF-interacting proteins in HepG2 cells were similar when cells were cultured in the presence or absence of insulin (FIG. 8D). As shown in FIG. 1E, in insulin-treated cells, USF-1 preferentially coimmunoprecipitated with those proteins that were found to be bound to the lipogenic promoters in the fed condition, whereas in the absence of insulin, USF-1 preferentially interacted with HDAC9.

To further address whether the binding of the various interacting proteins to the FAS promoter is USF dependent, ChIP was performed in transgenic mice containing CAT driven by the −444 FAS promoter with a specific mutation at the USF-binding site of −65 E box (−444 (−65 m)). Applicants previously showed that, due to the loss of the critical −65 E box where USF binds, the −444 (−65 m) FAS promoter does not have any activity, although the promoter contains an additional USF-binding site at −332 (Latasa et al., 2003). Applicants did not detect binding of any of the USF-1-interacting proteins to this FAS promoter containing the −65 E box mutation, even though USF-1 was bound to the −332 E box in both fasted and fed states (FIG. 1F, left panel). Furthermore, siRNA-mediated knockdown of USF-1 prevented recruitment of the USF-1-interacting proteins to the wildtype FAS promoter (FIG. 1F, right panel). Taken together, these data clearly demonstrate the requirement of USF-1 binding to the −65 E box for recruitment of various proteins to the FAS promoter.

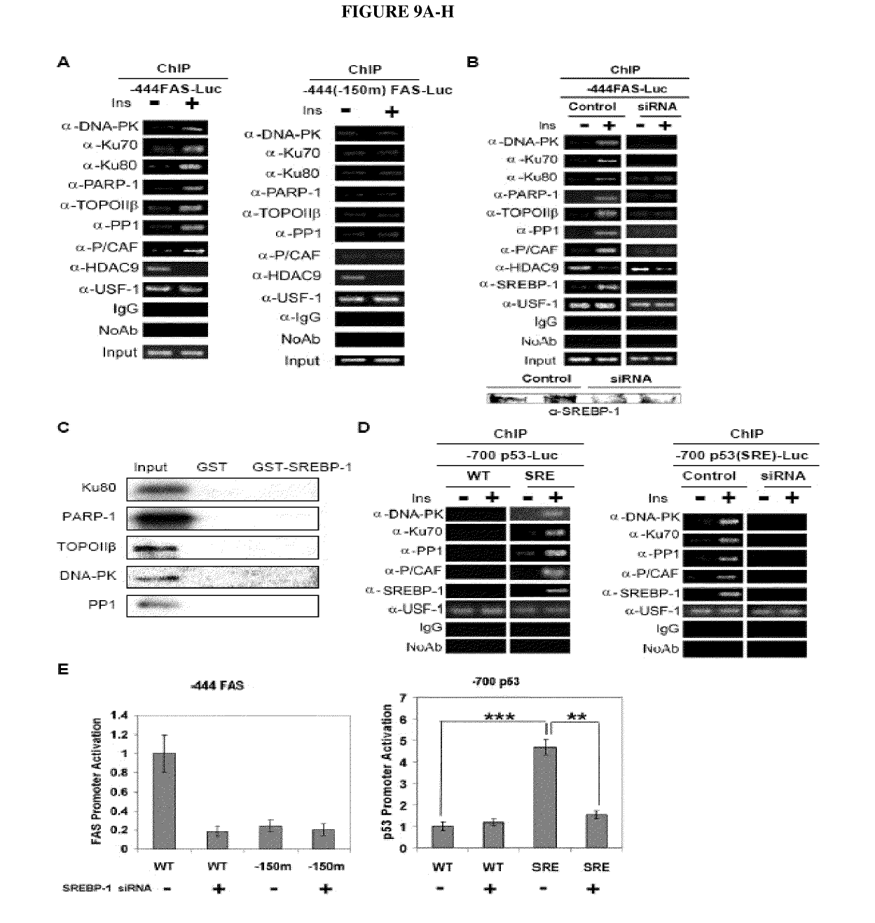

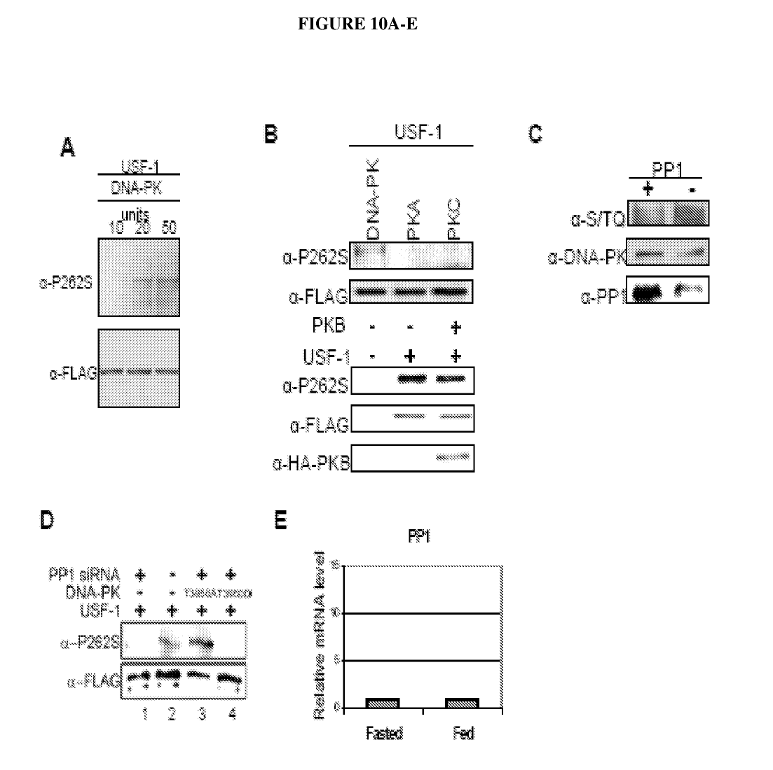

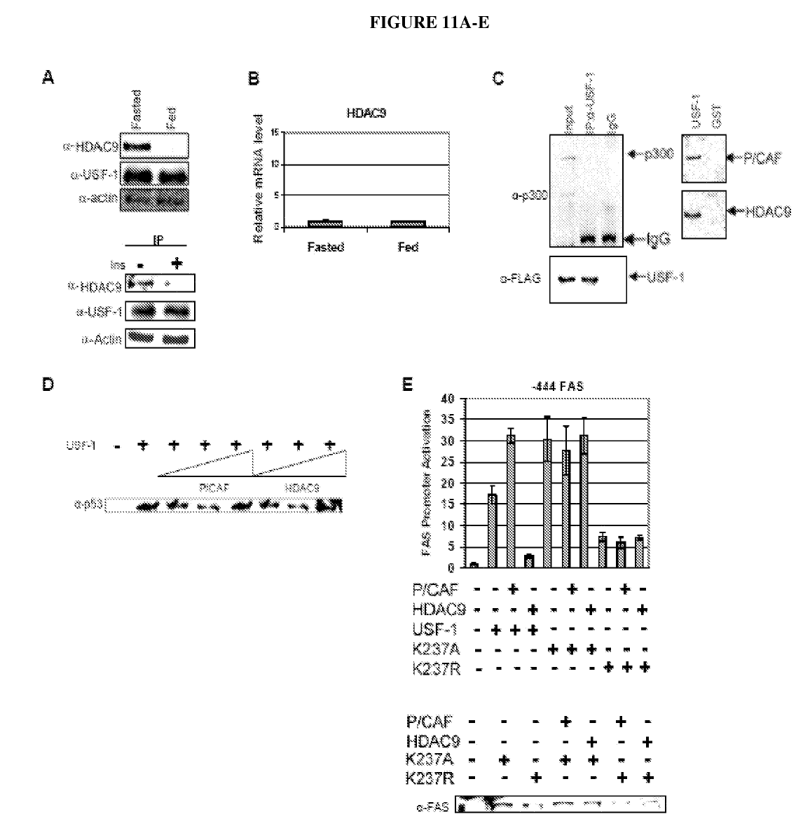

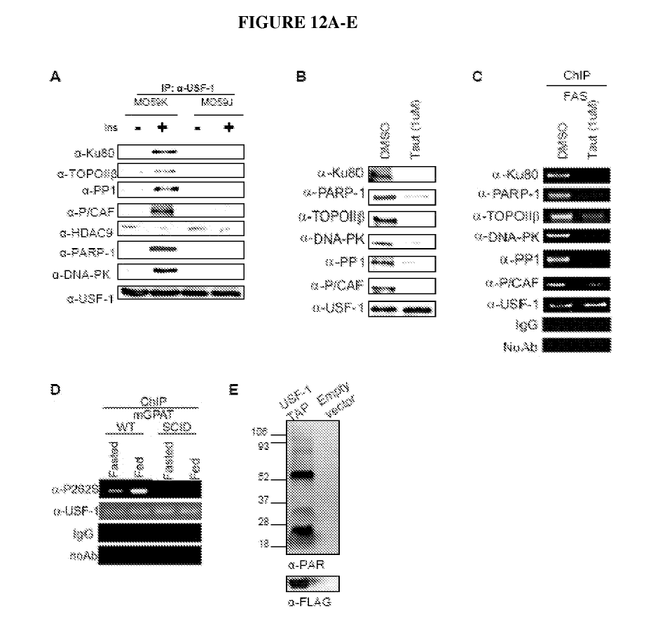

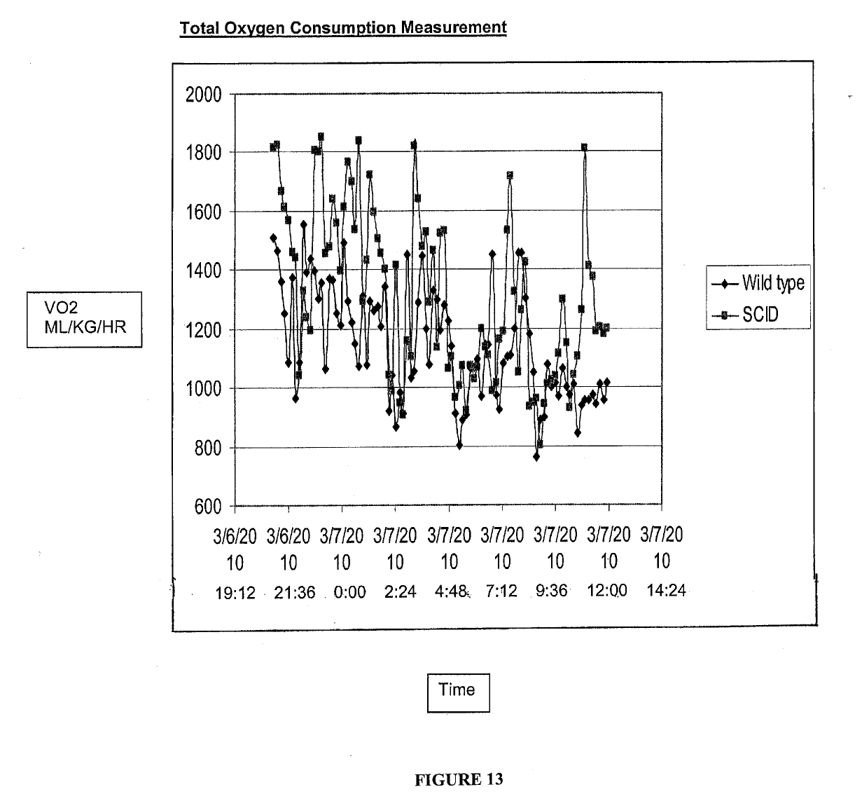

Because USF binding to the E box is necessary for SREBP binding to the nearby SRE in lipogenic promoters and USF and SREBP-1 directly interact for promoter activation (Latasa et al., 2003; Griffin et al., 2007), we examined whether the binding of the USF-1-interacting proteins to the FAS promoter is dependent on the SREBP-1 binding to SRE. Applicants performed ChIP in transgenic mice containing CAT driven by the −444 FAS promoter with a specific mutation at the −150SRE (−444 (−150 m)). As shown in FIG. 1G, Applicants could not detect recruitment of the various interacting proteins to the FAS promoter containing the −150 SRE mutation during feeding. Similar results were observed in HepG2 cells when transfected with −444 (−150 m) FAS-Luc or SREBP-1 siRNA (FIGS. 9A and 9B), correlating with the diminished FAS promoter activation (FIG. 9E). As a control, the p53 promoter was examined, which has a proximal E box but does not respond to feeding/insulin (FIGS. 8C and 9D). Upon insertion of an artificial SRE, the p53 promoter was activated by USF-1 recruiting various interacting proteins in response to insulin (FIGS. 9D and 9E), demonstrating that nearby SRE is critical for USF-1 to recruit various interacting proteins.

As shown, the components of DNA break/repair machinery were recruited to the FAS promoter in fed state. In this regard, it has recently been reported that a transient DNA break is required for estrogen receptor-regulated transcription (Juet al., 2006). By end labeling using biotin-UTP and subsequent ChIP, we clearly detected DNA breaks in the −444 FAS-CAT as well as the endogenous FAS promoters after 3 hr of feeding, a time point when binding of DNA-PK and TopoIIβ was detected (FIG. 1H). The observed DNA breaks in the FAS promoter region preceded the maximal FAS transcription that occurs 6 hr after the start of feeding (Paulauskis and Sul, 1989).

### Example II

### Feeding-Induced Phosphorylation of USF-1

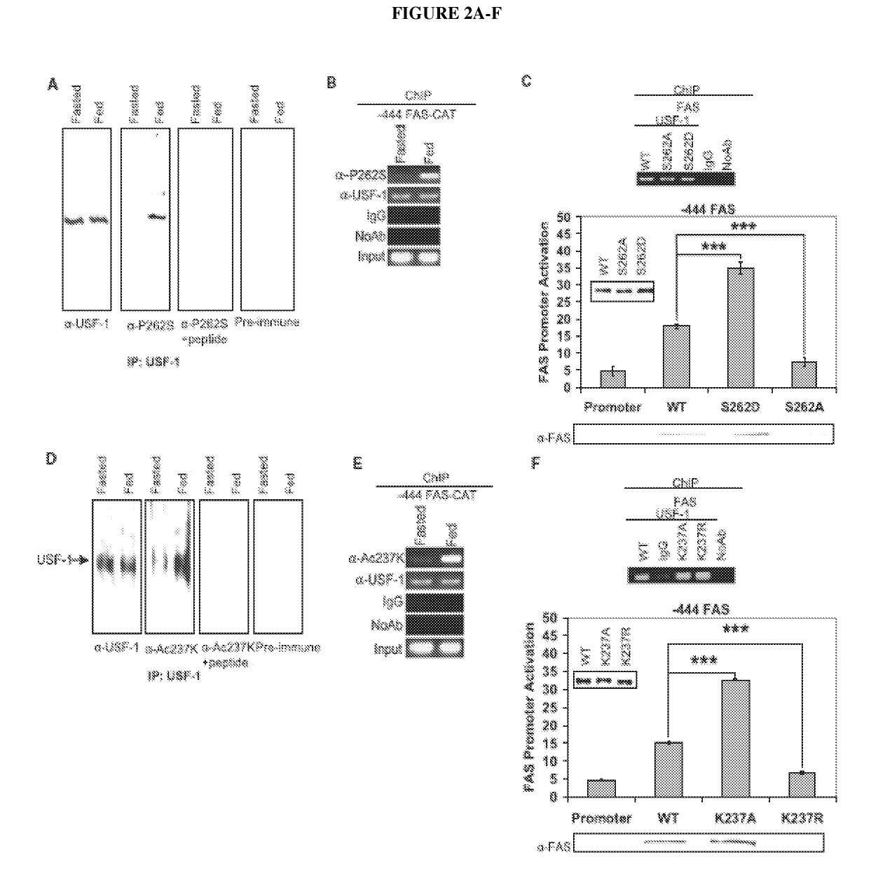

Constitutive binding of USF-1, despite its differential recruitments during fasting/feeding, prompted us to investigate whether USF-1 is posttranslationally modified. Applicants' immunoprecipitated USF-1 from liver nuclear extracts of fasted or fed mice and performed MS analysis. Notably, a phosphoserine residue was detected at the S262 of USF-1 only in nuclear extracts from fed mice. Applicants detected higher S262 phosphorylation of USF-1 in the fed state than in the fasted state (FIG. 2A, panel 2) using antibodies against a USF-1 peptide containing phosphorylated S262 (referred to as anti-P-USF-1) that Applicants generated. ChIP analysis of the FAS-CAT promoter using anti-P-USF-1 showed that this specific phosphoUSF-1 occupied the FAS promoter only in the fed state, even though USF-1 occupancy was detected in both fasted and fed conditions (FIG. 2B). Similarly, USF-1 bound to the mGPAT promoter was phosphorylated at S262 in fed state (FIG. 12D). To test the functional significance of this S262 phosphorylation, we expressed FLAGtagged-USF-1 containing a mutation at the S262 (S262D or S262A). Similar protein levels, were detected, of transfected S262 mutants and wild-type (WT) USF-1 (FIG. 2C, bottom panel). ChIP analysis of the FAS promoter using anti-FLAG antibodies showed no differences in promoter occupancy between WT and FLAG-tagged USF-1 proteins harboring S262 mutation (FIG. 2C, top panel). However, the S262D mutant that mimics hyperphosphorylation activated the FAS promoter at a much higher level than WT USF-1, whereas the nonphosphorylatable S262A mutant could no longer activate the FAS promoter (FIG. 2C, bottom panel). By immunoblotting lysates from these cells, we also detected changes in FAS protein levels corresponding to the FAS promoter activity (FIG. 2C, bottom panel). Taken together, these data suggest that the feeding-dependent phosphorylation of USF-1 at S262 is linked to FAS promoter activation.

### Example III

### Feeding-Induced Acetylation of USF-1

As shown in FIG. 1, USF-1-interacting proteins HDAC9 and P/CAF occupied the lipogenic gene promoters in fasted and fed states, respectively. During the MS analysis of USF-1 for posttranslational modification(s), we identified two acetylated lysine residues at K237 and K246 of USF-1. However, when MS analysis of immunoprecipitates was performed from cells co-transfected with USF-1 and P/CAF that interacts with USF in the fed state, acetylation of only K237, but not K246 was detected. Therefore, Applicants raised antibodies against USF-1 peptide containing acetylated K237 (anti-Ac-USF-1) and used them to compare acetylation of USF-1 at K237 in fasted and fed states. Indeed, Applicants detected higher K237 acetylation of USF-1 in the fed state (FIG. 2D, panel 2) compared to the fasted state. ChIP analysis of the FAS-CAT promoter using anti-Ac-USF-1 showed that the USF-1 bound to the FAS promoter was acetylated at K237 only in the fed state, even though USF-1 was bound to the FAS promoter in both fasted and fed states (FIG. 2E). These data indicate that K237 is likely to be a regulatory site of USF-1 during fasting/feeding and that its acetylation might be catalyzed by P/CAF in the fed state.

To test the functional effects of this putative acetylation site, FLAG-tagged USF-1 was expressed with a mutation at the K237 (K237A or K237R) in 293 cells. ChIP analysis of the FAS promoter using anti-FLAG antibodies showed no difference in recruitment among WT USF-1, FLAG-tagged USF-1 with the K237A mutation that mimics hyperacetylation, and the FLAG-tagged USF-1 with nonacetylatable K237R mutation (FIG. 2F, top panel). However, in the FAS promoter-reporter assay, cotransfection of the K237A mutant activated the FAS promoter at a much higher level than WT USF-1, whereas 237R mutant could no longer activate the FAS promoter (FIG. 2F, bottom panel). These differences in promoter activation were reflected in FAS protein levels upon immunoblotting of cell lysates (FIG. 2F, bottom panel). These data suggest that the feeding-dependent acetylation of USF-1 is responsible for FAS promoter activation in the fed condition.

### Example IV

### DNA-PK Mediates Feeding-Dependent Phosphorylation of USF-1

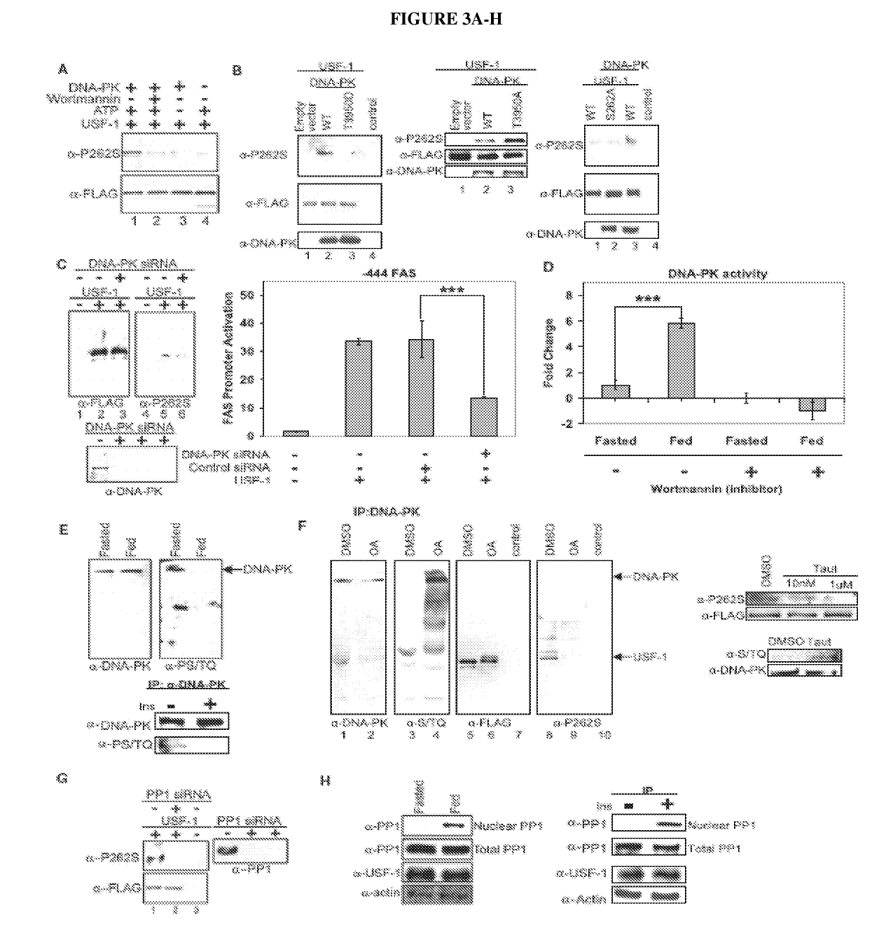

The first step in understanding how the feeding-dependent phosphorylation of USF-1 activates the FAS promoter would be to identify the kinase that catalyzes this S262 phosphorylation. A search of numerous phosphoprotein databases predicted that a member of the PIKK family of kinases likely phosphorylates the S262 site. DNA-PK is a multimeric nuclear serine/threonine protein kinase composed of the DNA-PK catalytic subunit and the Ku70/Ku80 regulatory subunits (Collis et al., 2005). Applicants found all of the DNA-PK subunits to be the USF-1-interacting proteins and bound to the FAS promoter in the fed state. Therefore, to examine whether S262 of USF-1 is a target of DNA-PK, in vitro phosphorylation of bacterially expressed USF-1 by DNA-PK was performed. Indeed, one could easily detect S262 phosphorylation of USF-1 by DNA-PK (FIG. 3A, lane 1) in vitro, which is DNA-PK concentration dependent (FIG. 10A). S262 phosphorylation was abolished when wortmannin was added at a concentration (Hashimoto et al., 2003) effective to inhibit DNA-PK activity (FIG. 3A, lane 2). However, S262 phosphorylation by PKA or PKC in vitro could not be detected, and changes in phosphorylation upon cotransfection with PKB could not be detected (FIG. 10B). Based on these results and the fact that DNA-PK is associated with USF-1 in the fed state, it was concluded that the S262 of USF-1 is a specific target of DNA-PK.

Next S262 phosphorylation of USF-1 by DNA-PK in cultured cells was tested. Applicants overexpressed USF-1 along with WT DNA-PK, kinase-dead DNA-PK with a T3950D mutation, or constitutive active DNA-PK with a T3950A mutation. T3950D mutation mimics hyperphosphorylation (Douglas et al., 2007), whereas T3950A mutation mimics dephosphorylation. Applicants detected higher S262 phosphorylation of USF-1 immunoprecipitated from cells overexpressing WT DNA-PK (FIG. 3B, left panel, lane 2), but not from cells expressing DNA-PK with T3950D mutation (FIG. 3B, lane 3) or control cells (FIG. 3B, lane 1). Furthermore, Applicants detected even higher S262 phosphorylation of USF-1 from cells expressing DNAPK with T3950A mutation compared to WT DNA-PK-expressing cells (FIG. 3B, middle panel, lane 3). Next, to investigate whether DNA-PK-mediated phosphorylation of USF-1 is S262 specific, Applicants overexpressed WT USF-1 or the S262A mutant along with DNA-PK. WT USF-1, but not USF-1 containing S262A mutation, was detected to have higher phosphorylation upon cotransfection with DNA-PK (FIG. 3B, right panel, lanes 2 and 3). To further verify the role of DNA-PK in S262 phosphorylation, siRNA-mediated knockdown of DNA-PK was performed. Applicants observed low but detectable S262 phosphorylation of USF-1 (FIG. 3C, left panel, lane 5). S262 phosphorylation was significantly reduced in the DNA-PK siRNA-transfected cells that had more than an 80% decrease in DNA-PK levels (FIG. 3C, lane 6). FAS promoter activity in DNA-PK siRNAtransfected cells was reduced by 65% compared to control siRNA-transfected cells (FIG. 3C, right panel), which was similar to that observed upon transfection of nonphosphorylatable S262A USF-1 mutant (FIG. 2C). These results demonstrate that S262 phosphorylation of USF-1 is mediated by DNA-PK.

### Example V

### PP1-Mediated Dephosphorylation/Activation of DNA-PK Causes USF-1 Phosphorylation Upon Feeding

Applicants found that DNA-PK phosphorylates USF-1 at S262 and that S262 phosphorylation is lower in the fasted state but increases upon feeding. This prompted us to ask whether the changes in DNA-PK activity account for the differences in S262 phosphorylation during fasting/feeding. Using the specific DNA-PK substrate, a biotinylated p53 peptide, DNA-PK activity in liver nuclear extracts of fasted or fed mice were compared (FIG. 3D). While total DNA-PK protein levels remained the same (data now shown), DNA-PK activity in the fed state was 6-fold higher than in the fasted state. Wortmannin treatment drastically reduced DNA-PK activity when measured with the DNA-PK-specific peptide as a substrate (FIG. 3D). This demonstrates that the kinase activity that was detected can be attributed to DNA-PK.

DNA-PK activity is known to be regulated by phosphorylation/dephosphorylation, independent of its activation by DNA. Thus, autophosphorylation of DNA-PK results in a decrease in its kinase activity, whereas dephosphorylation by PP1 activates DNA-PK (Douglas et al., 2001, 2007). Among the PIKK family members, DNA-PK is the only kinase that is activated by dephosphorylation. To examine the involvement of DNAPK in USF phosphorylation, we first examined the phosphorylation status of DNA-PK in fasted and fed states. DNA-PK phosphorylation was detected using phosphoserine/threonine antibodies that detect autophosphorylation at the S/TQ motifs of DNA-PK. As shown in the top panel of FIG. 3E, phosphorylation of DNA-PK was higher in the fasted state than in the fed state, whereas DNA-PK protein levels did not change. It was also found that DNA-PK phosphorylation was not detectable in insulin-treated HepG2 cells, whereas phosphorylation was easily detected in noninsulin-treated cells (FIG. 3E, bottom panel).

During the examination of the occupancy of USF-interacting proteins, it was found that PP1 along with DNA-PK was bound to lipogenic gene promoters in the fed state (FIG. 1C) when lipogenesis is induced. It is possible that PP1, which was found to be a USF-interacting protein, mediates the feeding/insulin signal by dephosphorylating DNA-PK. Therefore, Applicants tested the S262 phosphorylation status of USF-1 upon treatment with okadaic acid (OA), which is known to prevent dephosphorylation of DNA-PK (Douglas et al., 2001). As expected, phosphorylation of DNA-PK greatly increased in OA-treated cells (FIG. 3F, left panel, lane 4), whereas DNA-PK autophosphorylation was reduced in cells overexpressing PPlg (FIG. 10C). Applicants next examined S262 phosphorylation in OA-treated cells by western blotting of immunoprecipitated USF-1 with anti-FLAG or anti-P-USF-1 antibodies. Compared to a single USF-1 band detected in control DMSO-treated cells, several USF-1 bands were detected in OA-treated cells, suggesting a multisite phosphorylation of USF-1 (FIG. 3F, lane 6). However, S262 phosphorylation of USF-1 that was easily detected in control cells was hardly detectable in OA-treated cells (FIG. 3F, lane 9). To further test the specificity of PP1 on S262 phosphorylation status, tautomycin (Taut) was also used, which is known to more selectively inhibit PP1. As expected, Applicants easily detected phosphorylated DNA-PK in cells treated with Taut at 1 uM, but not in control cells (FIG. 3F, right panel). On the other hand, S262 phosphorylation of USF-1 was detected in control cells as expected but was decreased in cells treated with Taut at 10 nM and was hardly detectable at 1 uM (FIG. 3F, right panel). The role of PP1 was also tested by using a siRNA approach. S262 phosphorylation of USF-1 did not increase but, rather, greatly decreased in PP1 knockdown cells (FIG. 3G, lane 2), indicating that PP1 does not directly dephosphorylate S262 phosphorylation. Furthermore, S262 phosphorylation could be restored upon cotransfection of constitutively active DNA-PK (FIG. 10D). This indicates that S262 phosphorylation is through DNA-PK that is first dephosphorylated/activated by PP1. When the abundance of PP1 in liver nuclear extracts was compared, higher levels of PP1 were detected in the nucleus in the fed state than in the fasted state, whereas PP1 protein levels in total cell lysates as well as PP1 gene expression levels did not change (FIG. 3H, left panel and FIG. 10E). Similarly, PP1 was not detected in nuclear extracts from control HepG2 cells but was increased upon insulin treatment (FIG. 3H, right panel). Overall, it was concluded that the feeding-dependent S262 phosphorylation of USF-1 is mediated by DNA-PK. But first, DNA-PK is dephosphorylated/activated by PP1 whose level in nucleus increases in response to feeding/insulin.

### Example VI

### P/CAF-Mediated Acetylation of USF-1 Activates the FAS Promoter, Whereas HDAC9-Mediated Deacetylation Causes Promoter Inactivation

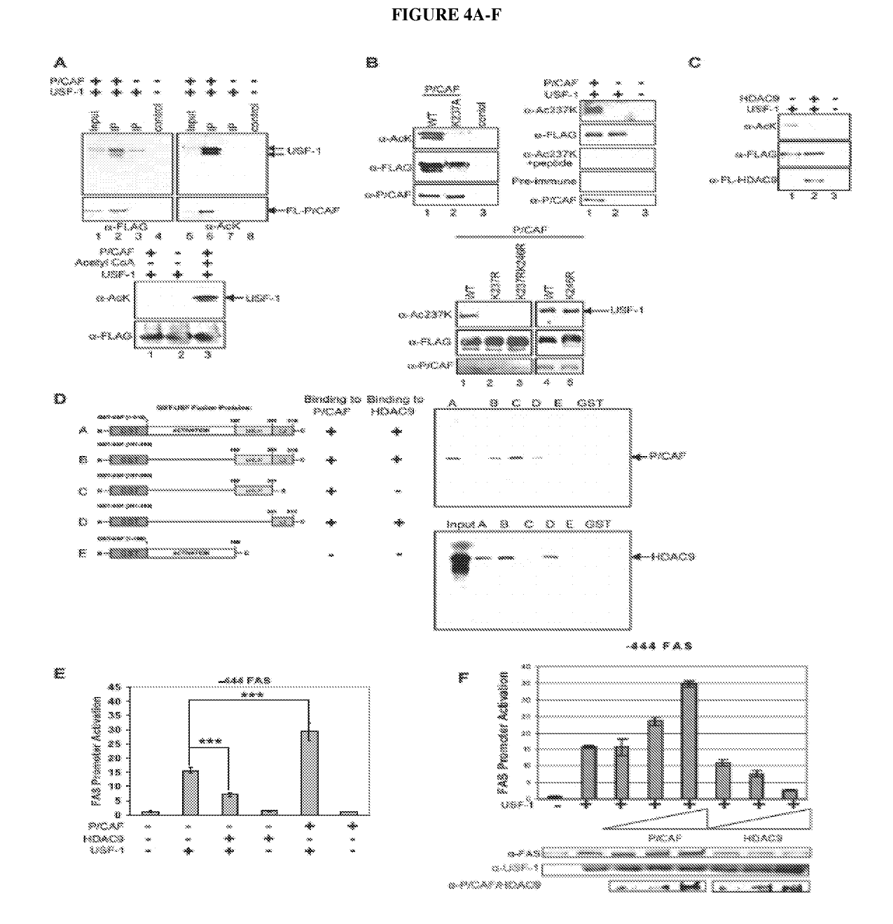

HDAC9 and P/CAF are recruited by and interact with USF-1 in a fasting/feeding-dependent manner. Therefore, Applicants next examined whether acetylation and deacetylation of USF-1 is through P/CAF and HDAC9, respectively. When Applicants cotransfected USF-1 and P/CAF, by using pan-acetyl lysine antibodies, higher acetylation of USF-1 was detected (FIG. 4A, top panel, lane 6). As shown in the bottom panel of FIG. 4A, USF-1 was acetylated in vitro by P/CAF (lane 3), and acetylation was not detected in the absence of P/CAF or acetyl CoA (lane 1 and 2). MS analysis of USF-1 in cells overexpressing P/CAF revealed a regulatory site at K237, the residue that was acetylated upon feeding (FIG. 2). To examine whether this site was a target of P/CAF, FLAG-tagged WT USF-1 or USF-1 mutated at K237 along with P/CAF was overexpressed. As detected by pan-acetyl lysine antibodies, only WT USF-1 was efficiently acetylated by P/CAF (FIG. 4B, top-left panel, lane 1), but the K237A USF-1 mutant was not (FIG. 4B, top-left panel, lane 2). Next anti-Ac-USF-1 antibodies specific for USF-1 acetylated at K237 were employed, and we detected higher K237 acetylation in cells overexpressing P/CAF (FIG. 4B, top right, lane 1). To further investigate whether P/CAF-mediated acetylation of USF-1 is K237 specific, WT USF-1 and various (K237 and K246) USF-1 mutants along with P/CAF were overexpressed. WT and K246R (FIG. 4B, bottom panel, lanes 1, 4, and 5), but not K237R or K237R/K246R (FIG. 4B, bottom panel, lanes 2 and 3), of USF-1 were found to be acetylated upon cotransfection with P/CAF, demonstrating that acetylation of K237, but not K246, is mediated by P/CAF.

With the binding of HDAC9 to the lipogenic promoters only in the fasted state, it was speculated that HDAC9 would be an ideal candidate to remove the P/CAF-mediated acetylation of USF-1 in the fed state. USF-1 and P/CAF along with HDAC9 or a control empty vector into 293 cells were transfected. A decrease in P/CAF-catalyzed acetylation of USF-1 in cells cotransfected with HDAC9 was detected (FIG. 4C, lane 2). Furthermore, significant HDAC9 protein levels in liver nuclear extracts from fasted, but not fed, mice or in nuclear extracts of HepG2 cells cultured in the absence, but not presence, of insulin were detected (FIG. 11A), whereas its expression did not change in various conditions (FIG. 11B). These experiments indicate that, in the fasted state, nuclear HDAC9 is in higher abundance and is recruited to the FAS promoter to deacetylate USF-1.

Applicants found by GST pull-down that USF-1 can directly interact with HDAC9 and P/CAF (but not p300) (FIG. 11C). Therefore, Applicants dissected the domains of USF-1 required for interaction with P/CAF and HDAC9. As shown in FIG. 4D, the bHLH domain of USF-1, the domain containing K237 that is acetylated by P/CAF, was sufficient for the interaction with P/CAF, although the leucine zipper (LZ) domain could weakly interact with P/CAF. On the other hand, for the USF-1 interaction with HDAC9, the LZ domain of USF-1 was sufficient for its interaction with HDAC9. Thus, the domains of USF-1 required for interaction are in proximity to K237, the residue modified by these HAT/HDAC.

Cotransfection of USF-1 together with HDAC9 resulted in a 50% decrease in FAS promoter activity in a fashion similar to that detected upon cotransfection of USF-1 containing a K237R mutation (FIGS. 2F and 4E). In contrast, the expression of USF-1 with P/CAF resulted in a 2-fold higher promoter activity in a manner similar to that observed upon cotransfection of USF-1 containing the K237A mutation (FIGS. 2F and 4E). Furthermore, cotransfection of P/CAF enhanced, while cotransfection of HDAC9 suppressed, USF-1 activation of the FAS promoter in a dose-dependent manner (FIG. 4F). Applicants detected changes in FAS protein levels parallel to the FAS promoter activity. In addition, cotransfecting P/CAF or HDAC9 with USF-1 containing K237A or K237R mutation did not change the FAS promoter activity or FAS protein levels (FIG. 11E). These data indicate that acetylation and deacetylation of USF-1 catalyzed by P/CAF and HDAC9, respectively, function as a dynamic switch for the transition between fasting/feeding in FAS promoter regulation.

### Example VII

### Phosphorylation-Dependent Acetylation of USF-1

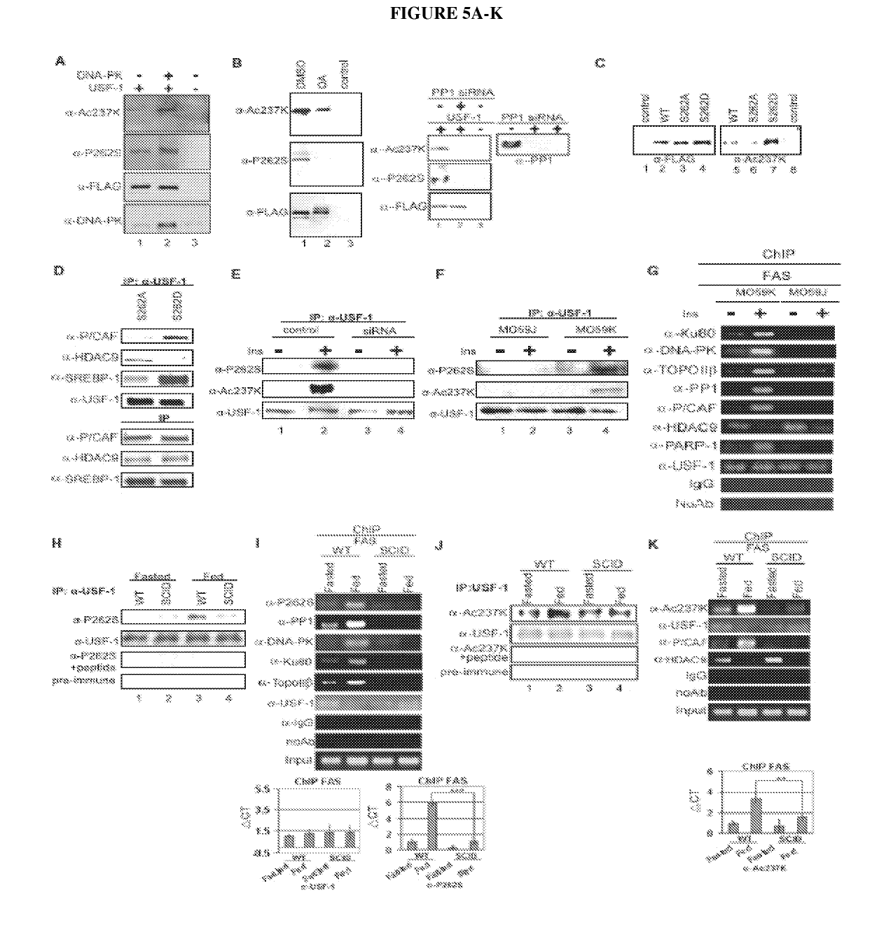

Since USF-1 is both phosphorylated and acetylated at nearby sites and these posttranslational modifications are critical for USF-1 function in FAS promoter activation, we tested whether an increase in S262 phosphorylation of USF-1 could affect K237 acetylation. USF-1 and DNA-PK were cotransfected and S262 phosphorylation and K237 acetylation of USF-1 was examined. If S262 phosphorylation affects acetylation, cotransfection of DNA-PK would cause not only S262 phosphorylation of USF-1, but also K237 acetylation. Indeed, S262 phosphorylation of USF-1 upon DNA-PK transfection strongly enhanced USF-1 acetylation at K237 (FIG. 5A, lane 2). Conversely, we detected a significant level of K237 acetylation of USF-1 in control cells, which was reduced in OA-treated cells (FIG. 5B, left panel, lane 2). Likewise, K237 acetylation of USF was high in control cells but was reduced to an undetectable level in PP1 siRNAtransfected cells (FIG. 5B, right panel, lane 1). Inactivation of PP1 by OA treatment or siRNA-mediated knockdown of PP1 caused phosphorylation/inactivation of DNA-PK resulting in reduced S262 phosphorylation of USF-1. This suggests that S262 phosphorylation brings about K237 acetylation. Applicants then asked whether phosphorylation of USF-1 at S262 could affect USF-1 acetylation status by transfecting FLAG-tagged WT USF-1 or S262 mutants and examining the K237 acetylation status of the various USF-1 forms. We found that the S262A mutant had the lowest K237 acetylation among the three USF-1 forms (FIG. 5C, lane 6), whereas the S262D mutant displayed the highest acetylation to a level significantly higher than WT USF-1 (FIG. 5C, lane 7). Overall, these results demonstrate phosphorylation-dependent acetylation of USF-1.

The simplest hypothesis underlying S262 phosphorylation-dependent acetylation of USF-1 would be that S262 phosphorylation/dephosphorylation affects recruitment of P/CAF and HDAC9, causing acetylation and deacetylation of K237 and USF-1, respectively. Communoprecipitation assay showed that the S262D mutant preferentially interacted with P/CAF in comparison to the S262A mutant (FIG. 5D). On the other hand, compared to the S262D mutant, the S262A mutant preferentially interacted with HDAC9, although the signal was low probably due to the low HDAC9 levels in the nucleus. We next examined whether S262 mutation of the USF-1 affects interaction of USF with SREBP-1 that we previously reported. We found that the S262D USF mutant, as compared to S262A mutant, preferentially interacted with SREBP-1. Taken together, these results show that the phosphorylation-dependent acetylation of USF-1 functions as a sensitive molecular switch, detecting nutritional status during the transition between fasting/feeding.

### Example VIII

### Feeding/Insulin-Dependent Phosphorylation/Acetylation of USF-1 are Diminished in DNA-PK Deficiency

To further demonstrate the requirement of DNA-PK in mediating the feeding/insulin-dependent phosphorylation/acetylation of USF-1, we transfected DNA-PK siRNA into HepG2 cells. Insulin treatment of these cells markedly increased S262 phosphorylation as well as K237 acetylation in control siRNA-transfected cells, whereas USF-1 levels remained the same (FIG. 5E, lanes 1 and 2). In contrast, insulin-mediated S262 phosphorylation/K237 acetylation of USF-1 in cells transfected with DNA-PK siRNA was markedly reduced and undetectable (FIG. 5E, lanes 3 and 4). We next compared the human glioblastoma cell line, M059J, which lacks DNA-PKcs and DNA-PK activity, and the related M059K cells containing WT DNA-PK (Feng et al., 2004) as a control. Treatment of M059K cells with insulin increased S262 phosphorylation and K237 acetylation of USF-1 (FIG. 5F, lanes 3 and 4), whereas insulin treatment of M059J cells did not result in any significant increase in USF modifications (FIG. 5F, lanes 1 and 2). These data demonstrate that DNA-PK is required not only for S262 phosphorylation, but also for K237 acetylation of USF-1 upon insulin treatment.

By ChIP, we also tested whether recruitment of various proteins to FAS promoter by USF is dependent on DNA-PK (FIG. 5G). Those proteins that were found to be bound to the lipogenic gene promoters in the fed condition were recruited by USF in insulin-treated M059K cells, but not in the DNA-PK deficient M059J cells. In the absence of insulin, HDAC9 was recruited by USF in both M059J and M059K cells, most likely because cytoplasmic export of HDAC9 was not affected by DNA-PK. Similarly, coimmunoprecipitation showed that USF-1 can interact better with various partners in insulin-treated M059K, but not in M059J cells (FIG. 12A). Furthermore, USF-1 interaction and recruitment of various proteins were abolished in 293 cells upon treatment with Taut that inhibits DNA-PK activity (FIGS. 12B and 12C). Overall, these results show that the recruitment of various proteins by USF-1 in feeding/insulin treatment is dependent on DNA-PK and DNA-PK-mediated S262 USF-1 phosphorylation.

Applicants next examined in vivo the DNA-PK-mediated and feeding-dependent S262 phosphorylation/K237 acetylation of USF-1 by employing DNA-PK-deficient SCID (severe combined immune deficiency) mice. A spontaneous mutation in the DNA-PK gene causes a 90% reduction of the protein in SCID mice (Danska et al., 1996), producing a phenotype highly reminiscent of DNA-PK null mice. Indeed, feeding-induced phosphorylation of USF-1 at S262 was greatly reduced in SCID mice compared to that observed in WT mice (FIG. 5H, lanes 4 and 3). ChIP analysis showed that the USF-1 detected on the FAS promoter in SCID mice in the fed state was not phosphorylated at S262 compared to the phosphoUSF-1 detected on the promoter in WT mice (FIG. 5I). Similarly, USF-1 bound to the mGPAT promoter was not phosphorylated at S262 in SOD mice in the fed state (FIG. 12D). Furthermore, we could not detect occupancy by DNA-PK, Ku80, TopoIIβ, and PP1 on the FAS promoter in SCID mice upon feeding (FIG. 5I). Because K237 acetylation of USF-1 is dependent on S262 phosphorylation as shown above, we investigated whether K237 acetylation was also reduced in SCID mice. We found that K237 acetylation upon feeding was greatly reduced in SCID mice compared to that detected in WT mice (FIG. 5J, lanes 4 and 2). The acetylated USF-1 bound to the FAS promoter in the fed state also was greatly reduced in SCID mice in ChIP analysis (FIG. 5K). This decrease in acetylated USF-1 bound to the FAS promoter could be explained by the decreased recruitment of P/CAF by USF-1 (FIG. 5K). HDAC9 binding was not different between WT and SCID mice probably because cytoplasmic export of HDAC9 was not affected in SCID mice. Overall, these results show in vivo the requirement of DNA-PK for S262 phosphorylation of USF-1 and for P/CAF-mediated K237 acetylation leading to transactivation of the FAS promoter.

### Example IX

### Feeding-Dependent Activation of the FAS Gene and De Novo Lipogenesis are Diminished in DNA-PK-Deficient SCID Mice

Because phosphorylation/acetylation of USF-1 for FAS promoter activation is through the PP1/DNA-PK-mediated signaling pathway, we assessed the transcriptional activation of the FAS gene in DNA-PK-deficient SCID mice during fasting/feeding. We first measured the nascent FAS RNA levels in liver nuclei from WT or SCID mice that were either fasted or fed (FIG. 6A) by RT-PCR. In WT mice, the FAS nascent RNA was not detectable in fasting but increased drastically upon feeding. On the other hand, the nascent FAS RNA was barely detectable in either fasted or fed SCID mice. RT-qPCR analysis indicated a 50-fold increase in FAS nascent transcript in WT mice upon feeding, whereas in SCID mice, the increase was 20-fold, representing approximately a 50%-60% decrease (FIG. 6B). Next, nuclear run-on assays using nuclei from WT and SCID mice upon feeding at various time points were performed. The rate of transcription measured by RT-qPCR of the newly extended nascent transcripts increased up to 10-fold in WT mice 6 hr after feeding, a result consistent with our previously published study. However, FAS transcription in SCID mice increased only by 6-fold, a 40% reduction compared to WT mice (FIG. 6C).

Because we observed transient DNA breaks in the FAS promoter region that preceded transcriptional activation upon feeding (FIG. 1I), we next examined whether the DNA break occurs in the FAS promoter region in SCID mice, but we could not detect transient DNA breaks, which we clearly detected in WT mice after 3 hr of feeding (FIG. 6D). Furthermore, in contrast to WT mice, ChIP analysis did not show binding of DNA-PK or TopoIIβ to the FAS promoter region in SCID mice. Because TopoIIβ catalyzes DNA breaks, the absence of DNA breaks in the FAS promoter region in SCID mice can be attributed to the impaired TopoIIβ recruitment that is dependent on the DNA-PK-catalyzed phosphorylation of USF-1. Thus, not only the diminished acetylation of USF-1, but also the impaired recruitment of the DNA break/repair components, which is dependent on USF-1 phosphorylation, probably contributed to the attenuated feeding-dependent transcriptional activation of the FAS gene in SCID mice. Overall, these results clearly show in vivo the critical role of DNA-PK in activation of FAS transcription by feeding.

We examined in vivo hepatic de novo lipogenesis in WT and SCID mice using a stable isotope method. Fractional de novo lipogenesis was hardly detected in fasting but was increased drastically during a 24 hr period of feeding in WT mice (FIG. 6E). However, feeding-induced fractional de novo lipogenesis was 60% lower in SCID mice after 24 hr of feeding compared to WT mice. To confirm that the decrease in de novo lipogenesis in SCID mice was due to a decrease in FAS induction, we examined the FAS protein levels in livers of WT and SCID mice after 24 hr of feeding. Indeed, FAS protein levels in SCID mice were significantly lower compared to WT mice (FIG. 6F). The hepatic triglyceride levels after 24 hr feeding were approximately 30% lower in SCID mice compared to WT mice; serum triglyceride levels were also significantly lower in SCID mice (FIG. 6G). Thus, impairment of feeding-dependent activation of FAS transcription in SCID mice leads to blunted induction of de novo lipogenesis, resulting in lower hepatic as well as—probably re-fleeting decreased VLDL secretion—serum triglyceride levels. In this regard, SCID mice also had a lower adipose tissue mass, indicative of a long-term defect in feeding induced lipogenesis (Table S1).

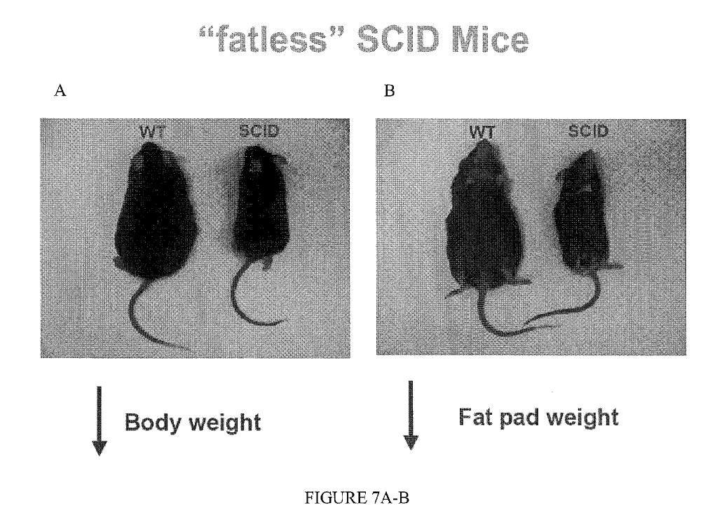

Referring to Table S1 (below), blood metabolite levels were measured from WT and SCID mice fasted for 40 hrs (top). No significant differences were found in blood glucose or serum insulin, NEFA and triglyceride levels between the two groups. Body weights and adipose and other organ weights of WT and SCID mice after 24 hrs feeding were measured (bottom). Body weights as well as weights of various fat depots expressed in percentage of body weight were lower in SCID mice compared to WT mice while no significant differences were detected in other organ weights. No significant differences were observed in food consumption between the two groups (3.8 g/day and 3.9 g/day for WT and SCID mice, respectively). Also see FIG. 7A-B, which shows a picture of WT compared to SCID mice for body weight.

TABLE S1

| | Glucose | Insullin | FFA | TG |
|---|---|---|---|---|
| Group | mg/dl | ng/ml | mEq/l | mg/dl |
| WT | 53.80 ± 9.30 | 0.21 ± 0.01 | 1.40 ± 0.20 | 60.20 ± 7.30 |
| SCID | 69.75 ± 7.54 | 0.22 ± 0.01 | 1.51 ± 0.05 | 60.45 ± 2.55 |

means ± SEM, n = 20

| | | Total Fat | Epididymal | | | | | | |
|---|---|---|---|---|---|---|---|---|---|
| | | Pad | Fat | Renal Fat | Inguinal Fat | Kidney | Heart | Liver | Spleen |
| Group | BW g | % Body Weight | | | | | | | |
| WT | 17.5 ± 0.5 | 0.60 ± 0.03 | 0.43 ± 0.03 | 0.07 ± 0.01 | 0.04 ± 0.01 | 1.31 ± 0.02 | 0.81 ± 0.04 | 6.60 ± 0.25 | 0.21 ± 0.03 |
| SCID | 15.0 ± 0.4* | 0.42 ± 0.08* | 0.28 ± 0.05* | 0.05 ± 0.01* | 0.05 ± 0.02 | 1.35 ± 0.04 | 0.86 ± 0.05 | 6.26 ± 0.10 | 0.16 ± 0.02 |

means ± SEM, n = 10, No significant differences in food consumption between groups

### Example X

### WT vs. SCID Mice Oxygen Consumption

Mice are housed in the metabolic cages for measurement of total oxygen consumption and results showed that SCID mice had a higher level of oxygen consumption over the course of 12 hrs compared to wild type mice indicating higher energy expenditure in these mice, contributing to the leanness to the SCID mice as shown in FIG. 13.

### REFERENCES

Allen, P. B., Kwon, Y. G., Nairn, A. C., and Greengard, P. (1998). Isolation and characterization of PNUTS, a putative protein phosphatase 1 nuclear targeting subunit. J. Biol. Chem. 273, 4089-4095.
Brady, M. J., and Saltiel, A. R. (2001). The role of protein phosphatase-1 in insulin action. Recent Prog. Horm. Res. 56, 157-173.
Casado, M., Vallet, V. S., Kahn, A., and Vaulont, S. (1999). Essential role in vivo of upstream stimulatory factors for a normal dietary response of the fatty acid synthase gene in the liver. J. Biol. Chem. 274, 2009-2013.
Collis, S. J., DeWeese, T. L., Jeggo, P. A., and Parker, A. R. (2005). The life and death of DNA-PK. Oncogene 24, 949-961.
Corre, S., and Galibert, M. D. (2005). Upstream stimulating factors: Highly versatile stress-responsive transcription factors. Pigment Cell Res. 18, 337-348.
Danska, J. S., Holland, D. P., Mariathasan, S., Williams, K. M., and Guidos, C. J. (1996). Biochemical and genetic defects in the DNA-dependent protein kinase in murine scid lymphocytes. Mol. Cell. Biol. 16, 5507-5517.
Dircks, L. K., and Sul, H. S. (1997). Mammalian mitochondrial glycerol-3-phosphate acyltransferase. Biochim. Biophys. Acta 1348, 17-26.
Douglas, P., Moorhead, G. B., Ye, R., and Lees-Miller, S. P. (2001). Protein phosphatases regulate DNA-dependent protein kinase activity. J. Biol. Chem. 276, 18992-18998.
Douglas, P., Cui, X., Block, W. D., Yu, Y., Gupta, S., Ding, Q., Ye, R., Morrice, N., Lees-Miller, S. P., and Meek, K. (2007). The DNA-dependent protein kinase catalytic subunit is phosphorylated in vivo on threonine 3950, a highly conserved amino acid in the protein kinase domain. Mol. Cell. Biol. 27, 1581-1591.
Engelman, J. A., Luo, J., and Cantley, L. C. (2006). The evolution of phosphatidylinositol 3-kinases as regulators of growth and metabolism. Nat. Rev. Genet. 7, 606-619.
Feng, J., Park, J., Cron, P., Hess, D., and Hemmings, B. A. (2004). Identification of a PKB/Akt hydrophobic motif Ser-473 kinase as DNA-dependent protein kinase. J. Biol. Chem. 279, 41189-41196.
Griffin, M. J., Wong, R. H., Pandya, N., and Sul, H. S. (2007). Direct interaction between USF and SREBP-1c mediates synergistic activation of the fatty acid synthase promoter. J. Biol. Chem. 282, 5453-5467.
Gu, W., and Roeder, R. G. (1997). Activation of p53 sequence-specific DNA binding by acetylation of the p53 C-terminal domain. Cell 90, 595-606.
Hashimoto, M., Rao, S., Tokuno, O., Yamamoto, K., Takata, M., Takeda, S., and Utsumi, H. (2003). DNA-PK: The major target for wortmannin-mediated radiosensitization by the inhibition of DSB repair via NHEJ pathway. J. Radiat. Res. (Tokyo) 44, 151-159.
Jerkins, A. A., Liu, W. R., Lee, S., and Sul, H. S. (1995). Characterization of the murine mitochondrial glycerol-3-phosphate acyltransferase promoter. J. Biol. Chem. 270, 1416-1421.
Ju, B. G., Lunyak, V. V., Perissi, V., Garcia-Bassets, I., Rose, D. W., Glass, C. K., and Rosenfeld, M. G. (2006). A topoisomerase II beta-mediated dsDNA break required for regulated transcription. Science 312, 1798-1802.
Latasa, M. J., Moon, Y. S., Kim, K. H., and Sul, H. S. (2000). Nutritional regulation of the fatty acid synthase promoter in vivo: Sterol regulatory element binding protein functions through an upstream region containing a sterol regulatory element. Proc. Natl. Acad. Sci. USA 97, 10619-10624.
Latasa, M. J., Griffin, M. J., Moon, Y. S., Kang, C., and Sul, H. S. (2003). Occupancy and function of the −150 sterol regulatory element and −65 E-box in nutritional regulation of the fatty acid synthase gene in living animals. Mol. Cell. Biol. 23, 5896-5907.
Martinez-Balbas, M. A., Bauer, U. M., Nielsen, S. J., Brehm, A., and Kouzarides, T. (2000). Regulation of E2F1 activity by acetylation. EMBO J. 19, 662-671.
Mejat, A., Ramond, F., Bassel-Duby, R., Khochbin, S., Olson, E. N., and Schaeffer, L. (2005). Histone deacetylase 9 couples neuronal activity to muscle chromatin acetylation and gene expression. Nat. Neurosci. 8, 313-321.
Moon, Y. S., Latasa, M. J., Kim, K. H., Wang, D., and Sul, H. S. (2000). Two 50-regions are required for nutritional and insulin regulation of the fatty-acid synthase promoter in transgenic mice. J. Biol. Chem. 275, 10121-10127.
Moustaid, N., and Sul, H. S. (1991). Regulation of expression of the fatty acid synthase gene in 3T3-L1 cells by differentiation and triiodothyronine. J. Biol. Chem. 266, 18550-18554.
Moustaid, N., Sakamoto, K., Clarke, S., Beyer, R. S., and Sul, H. S. (1993). Regulation of fatty acid synthase gene transcription. Sequences that confer a positive insulin effect and differentiation-dependent expression in 3T3-L1 preadipocytes are present in the 332 bp promoter. Biochem. J. 292, 767-772.
Moustaid, N., Beyer, R. S., and Sul, H. S. (1994). Identification of an insulin response element in the fatty acid synthase promoter. J. Biol. Chem. 269, 5629-5634.
Pajukanta, P., Lilja, H. E., Sinsheimer, J. S., Cantor, R. M., Lusis, A. J., Gentile, M., Duan, X. J., Soro-Paavonen, A., Naukkarinen, J., Saarela, J., et al. (2004). Familial combined hyperlipidemia is associated with upstream transcription factor 1 (USF1). Nat. Genet. 36, 371-376.
Paulauskis, J. D., and Sul, H. S. (1988). Cloning and expression of mouse fatty acid synthase and other specific mRNAs. Developmental and hormonal regulation in 3T3-L1 cells. J. Biol. Chem. 263, 7049-7054.
Paulauskis, J. D., and Sul, H. S. (1989). Hormonal regulation of mouse fatty acid synthase gene transcription in liver. J. Biol. Chem. 264, 574-577.
Printen, J. A., Brady, M. J., and Saltiel, A. R. (1997). PTG, a protein phosphatase 1-binding protein with a role in glycogen metabolism. Science 275, 1475-1478.
Sarbassov, D. D., Guertin, D. A., Ali, S. M., and Sabatini, D. M. (2005). Phosphorylation and regulation of Akt/PKB by the rictor-mTOR complex. Science 307, 1098-1101.
Sawadogo, M., and Roeder, R. G. (1985). Interaction of a gene-specific transcription factor with the adenovirus major late promoter upstream of the TATA box region. Cell 43, 165-175.
Shimomura, I., Bashmakov, Y., Ikemoto, S., Horton, J. D., Brown, M. S., and Goldstein, J. L. (1999). Insulin selectively increases SREBP-1c mRNA in the livers of rats with streptozotocin-induced diabetes. Proc. Natl. Acad. Sci. USA 96, 13656-13661.
Shin, D. H., Paulauskis, J. D., Moustaid, N., and Sul, H. S. (1991). Transcriptional regulation of p90 with sequence homology to Escherichia coli glycerol-3-phosphate acyltransferase. J. Biol. Chem. 266, 23834-23839.
Soncini, M., Yet, S. F., Moon, Y., Chun, J. Y., and Sul, H. S. (1995). Hormonal and nutritional control of the fatty acid synthase promoter in transgenic mice. J. Biol. Chem. 270, 30339-30343.
Sul, H. S., and Wang, D. (1998). Nutritional and hormonal regulation of enzymes in fat synthesis: studies of fatty acid synthase and mitochondrial glycerol-3-phosphate acyltransferase gene transcription. Annu. Rev. Nutr. 18, 331-351.
Sul, H. S., Latasa, M. J., Moon, Y., and Kim, K. H. (2000). Regulation of the fatty acid synthase promoter by insulin. J. Nutr. 130, 315S-320S.
Taniguchi, C. M., Emanuelli, B., and Kahn, C. R. (2006). Critical nodes in signaling pathways: Insights into insulin action. Nat. Rev. Mol. Cell Biol. 7, 85-96.
Wang, D., and Sul, H. S. (1995). Upstream stimulatory factors bind to insulin response sequence of the fatty acid synthase promoter. USF1 is regulated. J. Biol. Chem. 270, 28716-28722.
Wang, D., and Sul, H. S. (1997). Upstream stimulatory factor binding to the E-box at −65 is required for insulin regulation of the fatty acid synthase promoter. J. Biol. Chem. 272, 26367-26374.
Wang, D., and Sul, H. S. (1998). Insulin stimulation of the fatty acid synthase promoter is mediated by the phosphatidylinositol 3-kinase pathway. Involvement of protein kinase B/Akt. J. Biol. Chem. 273, 25420-25426.
West, A. G., Huang, S., Gaszner, M., Litt, M. D., and Felsenfeld, G. (2004). Recruitment of histone modifications by USF proteins at a vertebrate barrier element. Mol. Cell. 16, 453-463.
Yet, S. F., Lee, S., Hahm, Y. T., and Sul, H. S. (1993). Expression and identification of p90 as the murine mitochondrial glycerol-3-phosphate acyltransferase. Biochemistry 32, 9486-9491.
Yet, S. F., Moon, Y. K., and Sul, H. S. (1995). Purification and reconstitution of murine mitochondrial glycerol-3-phosphate acyltransferase. Functional expression in baculovirus-infected insect cells. Biochemistry 34, 7303-7310.
Andrews, N. C., and Faller, D. V. (1991). A rapid micropreparation technique for extraction of DNA-binding proteins from limiting numbers of mammalian cells. Nucleic Acids Res. 19, 2499.
Bertos, N. R., Wang, A. H., and Yang, X. J. (2001). Class II histone deacetylases: structure, function, and regulation. Biochem. Cell Biol. 79, 243-252.
Blunt, T., Gell, D., Fox, M., Taccioli, G. E., Lehmann, A. R., Jackson, S. P., and Jeggo, P. A. (1996). Identification of a nonsense mutation in the carboxyl-terminal region of DNA-dependent protein kinase catalytic subunit in the scid mouse. Proc. Natl. Acad. Sci. U.S.A. 93, 10285-10290.
Cohen, P. T. (2002). Protein phosphatase 1—targeted in many directions. J. Cell. Sci. 115, 241-256.
Coon, H., Xin, Y., Hopkins, P. N., Cawthon, R. M., Hasstedt, S. J., and Hunt, S. C. (2005). Upstream stimulatory factor 1 associated with familial combined hyperlipidemia, LDL cholesterol, and triglycerides. Hum. Genet. 117, 444-451.
Galibert, M. D., Carreira, S., and Goding, C. R. (2001). The Usf-1 transcription factor is a novel target for the stress-responsive p38 kinase and mediates UV-induced Tyrosinase expression. EMBO J. 20, 5022-5031.
Hosoi, Y., Miyachi, H., Matsumoto, Y., Ikehata, H., Komura, J., Ishii, K., Zhao, H. J., Yoshida, M., Takai, Y., Yamada, S., Suzuki, N., and Ono, T. (1998). A phosphatidylinositol 3-kinase inhibitor wortmannin induces radioresistant DNA synthesis and sensitizes cells to bleomycin and ionizing radiation. Int. J. Cancer 78, 642-647.
Ju, B. G., Solum, D., Song, E. J., Lee, K. J., Rose, D. W., Glass, C. K., and Rosenfeld, M. G. (2004). Activating the PARP-1 sensor component of the groucho/TLE1 corepressor complex mediates a CaMKinase IIdelta-dependent neurogenic gene activation pathway. Cell 119, 815-829.
Kim, Y. M., Watanabe, T., Allen, P. B., Kim, Y. M., Lee, S. J., Greengard, P., Nairn, A. C., and Kwon, Y. G. (2003). PNUTS, a protein phosphatase 1 (PP1) nuclear targeting subunit. Characterization of its PP1- and RNA-binding domains and regulation by phosphorylation. J. Biol. Chem. 278, 13819-13828.
Lesage, B., Beullens, M., Nuytten, M., Van Eynde, A., Keppens, S., Himpens, B., and Bollen, M. (2004). Interactor-mediated nuclear translocation and retention of protein phosphatase-1. J. Biol. Chem. 279, 55978-55984.
Li, B., Samanta, A., Song, X., Iacono, K. T., Bembas, K., Tao, R., Basu, S., Riley, J. L., Hancock, W. W., Shen, Y., Saouaf, S. J., and Greene, M. I. (2007). FOXP3 interactions with histone acetyltransferase and class II histone deacetylases are required for repression. Proc. Natl. Acad. Sci. U.S.A. 104, 4571-4576.
Lo, W. S., Trievel, R. C., Rojas, J. R., Duggan, L., Hsu, J. Y., Allis, C. D., Marmorstein, R., and Berger, S. L. (2000). Phosphorylation of serine 10 in histone H3 is functionally linked in vitro and in vivo to Gcn5-mediated acetylation at lysine 14. Mol. Cell 5, 917-926.
Patrone, G., Puppo, F., Cusano, R., Scaranari, M., Ceccherini, I., Puliti, A., and Ravazzolo, R. (2000). Nuclear run-on assay using biotin labeling, magnetic bead capture and analysis by fluorescence-based RT-PCR. BioTechniques 29, 1012-4, 1016-7.
Pavan Kumar, P., Purbey, P. K., Sinha, C. K., Notani, D., Limaye, A., Jayani, R. S., and Galande, S. (2006). Phosphorylation of SATB1, a global gene regulator, acts as a molecular switch regulating its transcriptional activity in vivo. Mol. Cell 22, 231-243.
Pavri, R., Lewis, B., Kim, T. K., Dilworth, F. J., Erdjument-Bromage, H., Tempst, P., de Murcia, G., Evans, R., Chambon, P., and Reinberg, D. (2005). PARP-1 determines specificity in a retinoid signaling pathway via direct modulation of mediator. Mol. Cell. 18, 83-96.
Reisman, D., and Rotter, V. (1993). The helix-loop-helix containing transcription factor USF binds to and transactivates the promoter of the p53 tumor suppressor gene. Nucleic Acids Res. 21, 345-350.
Schagger, H., Cramer, W. A., and von Jagow, G. (1994). Analysis of molecular masses and oligomeric states of protein complexes by blue native electrophoresis and isolation of membrane protein complexes by two-dimensional native electrophoresis. Anal. Biochem. 217, 220-230.
Sirito, M., Lin, Q., Maity, T., and Sawadogo, M. (1994). Ubiquitous expression of the 43- and 44-kDa forms of transcription factor USF in mammalian cells. Nucleic Acids Res. 22, 427-433.
Turner, S. M., Murphy, E. J., Neese, R. A., Antelo, F., Thomas, T., Agarwal, A., Go, C., and Hellerstein, M. K. (2003). Measurement of TG synthesis and turnover in vivo by 2H2O incorporation into the glycerol moiety and application of MIDA. Am J Physiol Endocrinol Metab 285(4), E790-803
Wang, A. H., Gregoire, S., Zika, E., Xiao, L., Li, C. S., Li, H., Wright, K. L., Ting, J. P., and Yang, X. J. (2005). Identification of the ankyrin repeat proteins ANKRA and RFXANK as novel partners of class Ha histone deacetylases. J. Biol. Chem. 280, 29117-29127.
Zhou, X., Marks, P. A., Rifkind, R. A., and Richon, V. M. (2001). Cloning and characterization of a histone deacetylase, HDAC9. Proc. Natl. Acad. Sci. U.S.A. 98, 10572-10577.
Sul H S, Smith S. Fatty acid synthesis in eukaryotes. In: Vance D E, Vance J E, eds. Biochemistry of Lipids, Lipoproteins and Membranes. 5th ed. Hungary: Elsevier 2008; 156.
Wang D, et al. J Biol Chem 1995; 270:28716-22.
Wang D, et al. J Biol Chem 1997; 272:26367-74.
Griffin M J, et al. J Biol Chem 2007; 282:5453-67.
Casado M, et al. J Biol Chem 1999; 274:2009-13.
Latasa M J, et al. Mol Cell Biol 2003; 23:5896-907.
Wang D, et al. J Biol Chem 1998; 273:25420-6.
Latasa M J, et al. Proc Natl Acad Sci USA 2000; 97:10619-24.
Kim J B, et al. J Clin Invest 1998; 101:1-9.
Wong R H, et al. Cell 2009; 136:1056-72.
Repa J J, et al. Genes Dev 2000; 14:2819-30.
Joseph S B, et al. J Biol Chem 2002; 277:11019-25.
Ishii S, et al. Proc Natl Acad Sci USA 2004; 101:15597-602.
Brady M J, et al. Recent Prog Horm Res 2001; 56:157-73.
Allen P B, et al. J Biol Chem 1998; 273:4089-95.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in molecular biology, genetics, or related fields are intended to be within the scope of the following claims.

1. A method, comprising:
a) providing:
i) a mammalian subject in need of treatment for a kidney disease;
ii) a DNA-PK inhibitor; and
b) administering to said subject said inhibitor under conditions to treat said disease.

2. A method, comprising:
a) providing:
i) a mammalian subject exhibiting symptoms of diabetes;
ii) a DNA-PK inhibitor; and
b) administering to said subject said inhibitor in an amount where at least one symptom is reduced.

3. A composition, comprising a DNA-PK inhibitor.

4. A method, comprising:
a) providing:
i) a mammalian subject in need of treatment for a metabolic disease;
ii) a DNA-PK inhibitor; and
b) administering to said subject said inhibitor under conditions to treat said disease.

5. A method, comprising:
a) providing:
i) a mammalian subject exhibiting symptoms of obesity;
ii) a DNA-PK inhibitor; and
b) administering to said subject said inhibitor in an amount where at least one symptom is reduced.

6. A method, comprising:
a) providing:
i) a mammalian subject in need of treatment for a kidney disease;
ii) a siRNA construct targeted to DNA-PK; and
b) administering said construct to said subject in an amount to treat said disease.


SEQUENCE LISTING (111 sequences)

SEQ ID NO: 1 - DNA, 21 bp, Artificial sequence
  Synthetic
catcaccatcttccaggagcg

SEQ ID NO: 2 - DNA, 20 bp, Artificial sequence
  Synthetic
tgaccttgcccacagccttg

SEQ ID NO: 3 - DNA, 21 bp, Artificial sequence
  Synthetic
gccaaagcgcattgttattcg

SEQ ID NO: 4 - DNA, 21 bp, Artificial sequence
  Synthetic
ggggtcactgttattagccac

SEQ ID NO: 5 - DNA, 21 bp, Artificial sequence
  Synthetic
tcctgcagcagcacttccgca

SEQ ID NO: 6 - DNA, 21 bp, Artificial sequence
  Synthetic
cagtgtaggtacagtgagctt

SEQ ID NO: 7 - DNA, 21 bp, Artificial sequence
  Synthetic
gctttccgggaggaggccatt

SEQ ID NO: 8 - DNA, 21 bp, Artificial sequence
  Synthetic
ctcttggattccccacacatc

SEQ ID NO: 9 - DNA, 21 bp, Artificial sequence
  Synthetic
ctgcaccagacaccacaaaac

SEQ ID NO: 10 - DNA, 21 bp, Artificial sequence
  Synthetic
ttccctggggaagccagtaag

SEQ ID NO: 11 - DNA, 21 bp, Artificial sequence
  Synthetic
agaggtagtgtgcttgaagga

SEQ ID NO: 12 - DNA, 21 bp, Artificial sequence
  Synthetic
ctctttaaggatgtctaccca

SEQ ID NO: 13 - DNA, 21 bp, Artificial sequence
  Synthetic
tggatgagaccctcatgtgtt

SEQ ID NO: 14 - DNA, 18 bp, Artificial sequence
  Synthetic
gagattagatgctgctat

SEQ ID NO: 15 - DNA, 21 bp, Artificial sequence
  Synthetic
gcacgccctggggatgaggtg

SEQ ID NO: 16 - DNA, 21 bp, Artificial sequence
  Synthetic
cgcagaataaagaatgtagcc

SEQ ID NO: 17 - DNA, 21 bp, Artificial sequence
  Synthetic
gtaaaggccgaggggcaaaga

SEQ ID NO: 18 - DNA, 21 bp, Artificial sequence
  Synthetic
aatgttcgtgctctttgggca

SEQ ID NO: 19 - DNA, 17 bp, Artificial sequence
  Synthetic
tgctcccagctgcaggc

SEQ ID NO: 20 - DNA, 20 bp, Artificial sequence
  Synthetic
gcccggtagctctgggtgta

SEQ ID NO: 21 - DNA, 21 bp, Artificial sequence
  Synthetic
ctgctagaagcctacagctct

SEQ ID NO: 22 - DNA, 21 bp, Artificial sequence
  Synthetic
cagcaccacaaaactcagaat

SEQ ID NO: 23 - DNA, 24 bp, Artificial sequence
  Synthetic
aaaggatgcccatgctacagagga

SEQ ID NO: 24 - DNA, 24 bp, Artificial sequence
  Synthetic
agtagactggcccttcttggtctt

SEQ ID NO: 25 - DNA, 23 bp, Artificial sequence
  Synthetic
gaccgagcgtggctacagcttca

SEQ ID NO: 26 - DNA, 22 bp, Artificial sequence
  Synthetic
ccgtcaggcagctcatagctct

SEQ ID NO: 27 - DNA, 26 bp, Artificial sequence
  Synthetic
acagccacactcacagagaatggggc

SEQ ID NO: 28 - DNA, 26 bp, Artificial sequence
  Synthetic
gaagaggcagactcggcgttccggag

SEQ ID NO: 29 - DNA, 21 bp, Artificial sequence
  Synthetic
gttatggcgactatccagctt

SEQ ID NO: 30 - DNA, 21 bp, Artificial sequence
  Synthetic
cccctaactgtagtcgctacc

SEQ ID NO: 31 - DNA, 21 bp, Artificial sequence
  Synthetic
gcacacgtggccccggcggac

SEQ ID NO: 32 - DNA, 18 bp, Artificial sequence
  Synthetic
gccacatgggctgacagc

SEQ ID NO: 33 - DNA, 19 bp, Artificial sequence
  Synthetic
cagccccgacgctcattgg

SEQ ID NO: 34 - DNA, 21 bp, Artificial sequence
  Synthetic
cttcatagccttatgcagttg

SEQ ID NO: 35 - DNA, 21 bp, Artificial sequence
  Synthetic
gacttttcacaaagcgttcct

SEQ ID NO: 36 - DNA, 21 bp, Artificial sequence
  Synthetic
agccagggtgagcacgtggga

SEQ ID NO: 37 - DNA, 21 bp, Artificial sequence
  Synthetic
gtggcatccatgaaactacat

SEQ ID NO: 38 - DNA, 21 bp, Artificial sequence
  Synthetic
gagccagagcagtaatctcct

SEQ ID NO: 39 - DNA, 21 bp, Artificial sequence
  Synthetic
acgtgacactgctgcgtgcca

SEQ ID NO: 40 - DNA, 21 bp, Artificial sequence
  Synthetic
atactcaggtgtcattctgtg

SEQ ID NO: 41 - DNA, 11 bp, Artificial sequence
  Synthetic
cctcaacccac

SEQ ID NO: 42 - DNA, 11 bp, Artificial sequence
  Synthetic
catcaccccac

SEQ ID NO: 43 - protein, 14 aa, Artificial sequence
  Synthetic
QELRQSNHRLSEEL

SEQ ID NO: 44 - protein, 14 aa, Artificial sequence
  Synthetic
CSMESTKSGQSKGG

SEQ ID NO: 45 - protein, 16 aa, Homo sapiens
KYVFRTENGGQVMYRV

SEQ ID NO: 46 - protein, 11 aa, Homo sapiens
KACDYIQELRQ

SEQ ID NO: 47 - protein, 16 aa, Homo sapiens
RQQVEDLKNKNLLLRA

SEQ ID NO: 48 - protein, 12 aa, Homo sapiens
RTENGGQVMYRV

SEQ ID NO: 49 - protein, 14 aa, Homo sapiens
RTHPYSPKSEAPRT

SEQ ID NO: 50 - protein, 21 aa, Homo sapiens
RLSEELQGLDQLQLDNDVLRQ

SEQ ID NO: 51 - protein, 29 aa, Homo sapiens
RLSEELQGLDQLQLDNVLRQQVEDLKNKN

SEQ ID NO: 52 - protein, 11 aa, Homo sapiens
RQQVEDLKNKN

SEQ ID NO: 53 - protein, 15 aa, Homo sapiens
RQQVEDLKNKLLLRA

SEQ ID NO: 54 - protein, 15 aa, Homo sapiens
RRDKINNWIVQLSKI

SEQ ID NO: 55 - protein, 14 aa, Homo sapiens
RDKINNWIVQLSKI

SEQ ID NO: 56 - protein, 13 aa, Homo sapiens
KINNWIVQLSKII

SEQ ID NO: 57 - protein, 12 aa, Homo sapiens
KINNWIVQLSKI

SEQ ID NO: 58 - protein, 17 aa, Homo sapiens
RILELDQFKGQQGQKRF

SEQ ID NO: 59 - protein, 19 aa, Homo sapiens
RIMLFTNEDNPHGNDSAKA

SEQ ID NO: 60 - protein, 19 aa, Homo sapiens
KAGDLRDTGIFLDLMHLKK

SEQ ID NO: 61 - protein, 21 aa, Homo sapiens
KTRTFNTSTGGLLLPSDTKRS

SEQ ID NO: 62 - protein, 19 aa, Homo sapiens
RTFNTSTGGLLLPSDTKRS

SEQ ID NO: 63 - protein, 13 aa, Homo sapiens
KCLEKEVAALCRY

SEQ ID NO: 64 - protein, 24 aa, Homo sapiens
RNLEALALDLMEPEQAVDLTLPKV

SEQ ID NO: 65 - protein, 30 aa, Homo sapiens
RNLEALALDLMEPEQAVDLTLPKVEAMNKR

SEQ ID NO: 66 - protein, 24 aa, Homo sapiens
RLGSLVDEFKELVYPPDYNPEGKV

SEQ ID NO: 67 - protein, 31 aa, Homo sapiens
RNLEALALDLMEPEQAVDLTLPKVEAMNKRL

SEQ ID NO: 68 - protein, 14 aa, Homo sapiens
KGTLGKFTVPMLKE

SEQ ID NO: 69 - protein, 16 aa, Homo sapiens
KSGLKKQELLEALTKH

SEQ ID NO: 70 - protein, 18 aa, Homo sapiens
RHLMLPDFDLLEDIESKI

SEQ ID NO: 71 - protein, 25 aa, Homo sapiens
KKYAPTEAQLNAVDALIDSMSLAKK

SEQ ID NO: 72 - protein, 24 aa, Homo sapiens
KYAPTEAQLNAVDALIDSMSLAKK

SEQ ID NO: 73 - protein, 10 aa, Homo sapiens
RLFQCLLHRA

SEQ ID NO: 74 - protein, 13 aa, Homo sapiens
KIKTLFPLIEAKK

SEQ ID NO: 75 - protein, 30 aa, Homo sapiens
KASFEEASNQLINHIEQFLDTNETPYFMKS

SEQ ID NO: 76 - protein, 13 aa, Homo sapiens
KCSESIPKDSLRM

SEQ ID NO: 77 - protein, 23 aa, Homo sapiens
KTAEAGGVTGKGQDGIGSKAEKT

SEQ ID NO: 78 - protein, 16 aa, Homo sapiens
KRKGDEVDGVDEVAKK

SEQ ID NO: 79 - protein, 17 aa, Homo sapiens
KVCSTNDLKELLIFNKQ

SEQ ID NO: 80 - protein, 17 aa, Homo sapiens
RVVSEDFLQDVSASTKS

SEQ ID NO: 81 - protein, 14 aa, Homo sapiens
KSKLPKPVQDLIKM

SEQ ID NO: 82 - protein, 16 aa, Homo sapiens
KKPPLLNNADSVQAKV

SEQ ID NO: 83 - protein, 13 aa, Homo sapiens
KGIPVVEHKVEKV

SEQ ID NO: 84 - protein, 18 aa, Homo sapiens
RRLHGLPEQFLYGTATKH

SEQ ID NO: 85 - protein, 17 aa, Homo sapiens
RLHGLPEQFLYGTATKH

SEQ ID NO: 86 - protein, 14 aa, Homo sapiens
RCGAALAGHQLIRG

SEQ ID NO: 87 - protein, 12 aa, Homo sapiens
RICSKPVVLPKG

SEQ ID NO: 88 - protein, 15 aa, Homo sapiens
RLYSLALHPNAFKRL

SEQ ID NO: 89 - protein, 16 aa, Homo sapiens
KWLLAHCGRPQTECRH

SEQ ID NO: 90 - protein, 12 aa, Homo sapiens
RFNNYVDCNKKF

SEQ ID NO: 91 - protein, 20 aa, Homo sapiens
KINQVFHGSCITEGNELTKT

SEQ ID NO: 92 - protein, 32 aa, Homo sapiens
RSSFDWLTGSSTDPLVDHTSPSSDSLLFAHKR

SEQ ID NO: 93 - protein, 33 aa, Homo sapiens
RSSFDWLTGSSTDPLVDHTSPSSDSLLFAHKRS

SEQ ID NO: 94 - protein, 15 aa, Homo sapiens
RLGLPGDEVDNKVKG

SEQ ID NO: 95 - protein, 20 aa, Homo sapiens
RLLQIIERYPEETLSLMTKE

SEQ ID NO: 96 - protein, 14 aa, Homo sapiens
KGANRTETVTSFRK

SEQ ID NO: 97 - protein, 21 aa, Homo sapiens
KKGGSWIQEINVAEKNWYPRQ

SEQ ID NO: 98 - protein, 12 aa, Homo sapiens
KNVQLQENEIRG

SEQ ID NO: 99 - protein, 13 aa, Homo sapiens
KIKYPENFFLLRG

SEQ ID NO: 100 - protein, 22 aa, Homo sapiens
KIFCCHGGLSPDLQSMEQIRRI

SEQ ID NO: 101 - protein, 20 aa, Homo sapiens
KTFTDCFNCLPIAAIVDEKI

SEQ ID NO: 102 - protein, 20 aa, Homo sapiens
KYGQFSGLNPGGRPITPPRN

SEQ ID NO: 103 - protein, 20 aa, Homo sapiens
KYGQFSGLNPGGRPITPPRN

SEQ ID NO: 104 - protein, 16 aa, Homo sapiens
KMTDSHVLEEAKKPRV

SEQ ID NO: 105 - protein, 21 aa, Homo sapiens
KHDILNFLTYADEYAIGYFKK

SEQ ID NO: 106 - protein, 22 aa, Homo sapiens
KHDILNFLTYADEYAIGYFKKQ

SEQ ID NO: 107 - protein, 23 aa, Homo sapiens
HYVGYIKDYEGATLMGCELNPRI

SEQ ID NO: 108 - protein, 17 aa, Homo sapiens
KSKEPRDPDQLYSTLKS

SEQ ID NO: 109 - protein, 16 aa, Homo sapiens
KSHQSAWPFMEPVKRT

SEQ ID NO: 110 - protein, 19 aa, Homo sapiens
RVFTNCKEYNPPESEYYKC

SEQ ID NO: 111 - protein, 19 aa, Homo sapiens
KQLQQELLLIQQQQQIQKQ